SEQUENCE LISTING (4 sequences)

SEQ ID NO: 1 - protein, 8 aa, Staphylococcus aureus
YSTCDFIM

SEQ ID NO: 2 - protein, 9 aa, Staphylococcus aureus
GVNACSSLF

SEQ ID NO: 3 - protein, 7 aa, Staphylococcus aureus
INCDFLL

SEQ ID NO: 4 - protein, 8 aa, Staphylococcus aureus
YSTCYFIM

(19) United States
(12) Patent Application Publication
(10) Pub. No.: US 2010/0143972 A1
(43) Pub. Date: Jun. 10, 2010

(54) Method of Making Cyclic Polypeptides with Inteins

(76) Inventor: Alexander R. Horswill, Coralville, IA (US)

Correspondence Address: FULBRIGHT & JAWORSKI L.L.P., 600 CONGRESS AVE., SUITE 2400, AUSTIN, TX, 78701, US

(21) Appl. No.: 12/519,283

(22) PCT Filed: Dec. 14, 2007
(86) PCT No.: PCT/US07/87663

§ 371 Date: Feb. 19, 2010

Related U.S. Application Data
(60) Provisional application No. 60/870,033, filed on Dec. 14, 2006.
(60) Provisional application No. 60/972,124, filed on Sep. 13, 2007.

(51) Int. Cl. C12P 21/06

(52) U.S. Cl. 435/69.7; 435/70.1

(57) ABSTRACT

Methods for producing cyclic polypeptides comprising a lactone or thiolactone ring. In certain cases, intein fusion proteins may be used in a method for biologically producing internally cyclized polypeptides. The new methods enable the construction of cyclic polypeptide libraries that may be screened for the biological activity of cyclic polypeptides. Methods provided may be used, for instance, to produce and optimize novel cyclic peptides for use in treating Staphylococcal infections.

The present invention claims benefit of priority to U.S. Provisional Application Ser. No. 60/972,124 filed Sep. 13, 2007, and U.S. Provisional Application Ser. No. 60/870,033, filed Dec. 14, 2006, the entire contents of both applications incorporated herein by reference.

### BACKGROUND OF THE INVENTION

1. Field of the Invention

The invention generally concerns methods for cyclic polypeptide synthesis and screening. More specifically biological methods for producing and screening internally cyclized polypeptides are provided.

2. Description of Related Art

Presently a great amount of research effort is being used to develop biologically active (e.g., therapeutic) proteins that may exhibit greater target specificity that corresponding small molecules. Due to the partial conformational freedom in peptide bonds biologically active peptides and polypeptides undergo conformational changes that are important for their activity. However, in some cases, polypeptides exhibit enhanced or novel activity when their conformation is constrained through an intramolecular covalent bond. Thus, such cyclic peptides constitute an import class of bioactive molecules that have a wide range of diagnostic and therapeutic applications. For example, many naturally occurring peptide antibiotics are internally cyclized, having a cyclic core with a covalently attached tail (e.g., bacitracin and arylomycin) (Ming and Epperson, 2002; Schimana et al., 2002), suggesting other cyclic peptides may function as antibacterial agents. Similarly, S. aureus utilizes modified peptides that are 8 to 9 amino acids in length with the carboxyl-terminal five amino acids constrained as a thiolactone (termed autoinducing peptides or AIPs) as the signal to regulate pathogenesis (Novick, 2003). Thus, the S. aureus AIPs are also being examined as therapeutic candidates for treating Staph infections involving bacteria that are resistant to conventional antibiotics. However, despite the promising potential of cyclic peptides, the cost and difficulty in chemical synthesis has limited there further development.

Chemical methods for the synthesis of cyclic peptides have recently been developed involving solid- and liquid-phase synthesis schemes (Camarero et al., 1998; Tam & Yu, 1998; Love et al., 1999). Furthermore, chemical synthesis methods have been shown to regenerate biologically active polypeptides such as S. aureus AIPs (Mayville et al., 1999). However, these methods still suffer from a number of major draw-backs. First, the methods are too complex and far too cumbersome to generate a diverse cyclic peptide library that could be used for screening. Furthermore, chemically synthesized cyclic polypeptides must be short in length to be efficiently synthesized. Finally, since chemical synthesis methods do not provide a genetically encoded polypeptide there is no efficient method for determining the structure of a peptide identified in a screening protocol. Thus, despite advances in chemical cyclic peptide synthesis, a biological method for high throughput cyclic polypeptide production would sorely needed.

Methods have been developed to generate certain types of cyclic peptides in vivo by using the catalytic activity of inteins (Kimura et al., 2006). Inteins are naturally-occurring enzymes that splice proteins together post-translationally (Perler, 2006). The most significant chemistry catalyzed by an intein is the first step in the splicing reaction, the conversion of a peptide bond to a thioester, and this activity may be exploited for additional applications (e.g., lactones and thiolactone bond formation). In some cases, for instance, inteins can be used to generate circular peptides in vivo (U.S. Patent Publication 2002/0177691). In these methods the two functional portions of an intein sequence are separated and positioned in reversed order with an intervening a peptide of interest. The result of these method is to produce a circular peptide sequence. Unfortunately, a many biologically active cyclic peptides, such as the S. aureus AIPs are not circular, but are rather internally cyclized to faun a lariat-like structure. Previously, a method biologically producing such an internally cyclized polypeptide had not been described.

### SUMMARY OF THE INVENTION

In a first embodiment the invention concerns biological methods for making internally cyclized polypeptides. As used here the phrase “internally cyclized polypeptide” refers to a polypeptide comprising a ring of covalently bonded amino acids with at least one additional amino acid extending from the ring. For example, an internally cyclized polypeptide may comprise one or more amino terminal amino acids linked via a peptide bond to a lactone or thiolactone amino acid ring. Thus, an internally cyclized polypeptide of the invention may be defined as having two regions the cyclic region and linear region joined to the cyclic region by a peptide bond. As used herein the term “linear region” is used merely for distinction for the cyclic region and refers to the amino acid(s) extending from the cyclic region of an internally cyclized polypeptide. Thus, it will be understood that a linear region of amino acids may be further modified in such a way as to mediate internal bonding (e.g., by intra- or intermolecular disulfide bonding). In some aspects, a method according to the invention may comprise producing at least a first internally cyclized polypeptide comprising, (a) expressing at least a first fusion protein in a cell, wherein the fusion protein comprises (i) a functional intein polypeptide fused to the carboxy-terminus of (ii) a polypeptide domain having at least one internal cysteine, threonine or serine residue and (c) exposing at least a first fusion protein to reducing conditions, thereby producing an internally cyclized polypeptide and a free intein polypeptide. In addition, the C-terminal asparagine residue of an intein may be mutated such that the splicing mechanism will terminate following the N—S acyl shift. Additionally, without a nucleophilic (cysteine, serine, threonine) residue at the beginning of a C-terminal extein, intein-mediated splicing will be unable to occur.

As used herein “a polypeptide domain having at least one internal cysteine or serine” refers to a stretch of amino acids that comprises at least one cysteine or serine residue at a position other then the amino or carboxy-terminus of the domain. Thus, in some cases, a polypeptide domain of the invention may be defined as comprising an amino acid other than cysteine or serine at the amino terminal position (e.g., methionine). In some cases a polypeptide domain comprising an internal cysteine or serine may be defined as comprising at least 5 amino acid positions. In some further cases such a domain may comprise 6, 7, 8, 9, 10, 15, 20, 25, 30, 35, 40, 45, 50 or more amino acid positions. Furthermore, in certain aspects, the position the internal cysteine or serine residue may be further defined as, for example, at least 2, 3, 4, 5, 6, 7, 8, 9, 10 or more amino acid positions away from the active intein polypeptide. The skilled artisan will recognize that the distance of the internal cysteine or serine from the carboxy-terminus of the domain (i.e., the distance from the intein polypeptide) will determine the number of amino acids that form the cyclic region of an internally cyclized polypeptide produced from the fusion protein. Thus, in some aspects, a method of the invention may be defined as a method for producing an internally cyclized polypeptide comprising at least or at most a 3, 4, 5, 6, 7, 8, 9, 10, 12, 13, 14, 15, 20, 25, 30, 35, 40, 45 or 50 amino acid cyclic region. Furthermore, the skilled artisan will understand that the number of amino acids a polypeptide domain that are position N-terminally relative to a cysteine or serine residue may determine the number of amino acids in the linear domain of the internally cyclized polypeptide. Hence, in certain further aspects, a method of the invention may be defined as a method for producing an internally cyclized polypeptide comprising a 3, 4, 5, 6, 7, 8, 9, 10 or more amino acid linear region.

In certain aspects of the invention, a polypeptide fused to the amino terminus of a functional intein polypeptide may comprise a single serine and no cysteine residues or conversely may comprise a single cysteine residue and no serine residues. However, in some aspects, a polypeptide domain that is fused to an intein polypeptide may comprise two or more serine and/or cysteine residues or may comprise at least one serine and at least one cysteine residue. In these aspects the skilled artisan will recognize that when exposed to reducing conditions more than one internally cyclized polypeptide may be formed due to alternative nucleophilic attack by two or more cysteine and/or serine residues. In these aspects two more species of internally cyclized polypeptides may be formed when the fusion protein is exposed to reducing conditions. In these aspects various species of cyclized polypeptide may be favored in the reaction by adjusting for example the temperature, pH, incubation time, the amino acid sequence of the cyclic domain (i.e., between a serine, threonine or cysteine residue and the intein) and/or concentration of reducing agent during the exposure of the fusion protein. Thus, in certain aspects the invention provides a method for selectively producing a species of internally cyclized polypeptides from an intein fusion protein of the invention by modulating the conditions under which the fusion protein is reduced.

In certain aspects methods and compositions of the invention involve an active intein polypeptide. As used herein the term “active intein polypeptide” refers to any intein or intein domain that is capable of catalyzing a N→S acyl shift to form a thioester at the amino-terminus of the intein domain. An intein according to the invention may be a naturally occurring intein domain (e.g., from a bacterial or yeast cell) or a genetically engineered artificial intein domain. For example, in some specific aspects, an intein polypeptide for use in the invention may be a Synechocystis (Ssp) DnaB, a Synechocystis DnaE (NCBI accession nos. 575328 and S76958), a Mycobacterium xenopi (Mxe) GyrA intein (NCBI accession no. P72065), Mycobacterium tuberculosis (Mtu) recA intein (NCBI accession nos. CAB02519.1 and CAA03856), Saccharomyces cerevisiae Vma intein (NCBI accession no. PXBYVA) or a catalytically active fragment thereof. Thus, in some very specific embodiments methods and compositions of the invention may employ an active domain of the Synechocystis DnaB intein (NCBI accession no. Q55418; Wu et al., 1998).

In some embodiments a fusion protein according to the invention may comprise additional amino acid sequences fused to the amino terminus or carboxy-terminus of the fusion protein. For example, in some cases, a fusion protein may comprise a secretion signal fused to the amino terminus that mediates secretion of the fusion protein (the internally cyclized polypeptide). Furthermore, a fusion protein may comprise an anchoring domain fused to amino or carboxy-terminus of the fusion protein. For example, an anchoring domain may be a membrane anchoring domain or a purification tag. A purification tag for use in the invention may be, for example, a chitin binding domain (CBD), a maltose binding protein (MBP), a polyhistadine tag or a glutathione S transferase (GST) tag. Furthermore, in certain aspects, a fusion protein of the invention may comprise additional amino acid sequences (fused to the N- or C-terminus of the fusion protein) that enable detection of the fusion protein or the internally cyclized polypeptide. For example, the fusion protein may comprise a detection tag such reporter polypeptide or an antibody epitope tag such as myc, or HA tag. In certain instances, a reporter polypeptide may be a as a fluorescent protein (e.g., green fluorescent protein (GFP)) or an enzyme such as luciferase. Furthermore, in certain aspects, additional domains of intein fusion proteins such as anchoring domains or detection tags may further comprise proteolytic cleavage sequences. In certain cases, such cleavage sequences enable additional domains (e.g., a purification tag) to be cleaved away from an intein fusion protein or cyclic polypeptide.

In still further embodiments of the invention there is provided a nucleic acid comprising expression cassette encoding a fusion protein comprising (i) a functional intein polypeptide fused to the carboxy-terminus of (ii) a polypeptide domain having at least one internal cysteine or serine residue. As used herein the term “expression cassette” refers to the nucleic acids encoding a fusion protein of the invention as well as sequences that mediate the expression of the fusion protein in a cell, for example an expression cassette may comprise a promoter, enhance, transcription termination signal, polyadenylation signal or other sequences that mediation fusion protein expression. Promoters for use in an expression cassette of the invention will depend upon the cell type in which the protein is to be expressed. For example, promoters may be eukaryotic or prokaryotic promoters. In some particular aspects, a promoter of the invention may be defined as an inducible promoter such as an IPTG inducible promoter. Thus, in some aspects, a method of the invention may involve transforming a cell with a nucleic acid comprising an intein fusion protein expression cassette and incubating said cell under conditions supporting expression of the fusion protein.

Various types of cells may be used for the expression of a fusion protein according to the invention. A cell for use in the invention may be a eukaryotic or prokaryotic cell, for example, a bacterial, yeast, insect, or mammalian cell. In some very specific aspects, a cell for use in the invention may be a Gram negative bacterial cell such as an E. coli cell. Furthermore, in certain cases, a cell of the invention may comprise an oxidizing cytoplasm such as a cytoplasm that lacks or has reduced amounts of thiols (e.g., glutathione). Cells having an oxidized cytoplasm may in some cases enable the production or purification of great amounts of intact intein fusion protein since thiols can mediated attack on the intein fusion protein thioester resulting in cleavage of the fusion protein. Certain mutations are known that render the cytoplasm of bacterial cells oxidized, for instance, in some cases a trxB1 mutant bacterial cell may be used according to the invention. TrxB1 mutant cells lack a functional glutathione system and yet remains viable under aerobic conditions (Karin et al., 2005).

Furthermore, in certain aspects, methods of the invention involve a step of (c) exposing at least a first fusion protein to reducing conditions, thereby producing an internally cyclized polypeptide and a free intein polypeptide. Thus, in some cases exposing a fusion protein to reducing conditions comprises exposing the fusion protein a reducing agent such a non-thiol reducing agent. In certain specific aspects, a non-thiol reducing agent for use in the invention may be comprise tris(2-carboxyethyl)phosphine hydrochloride (TCEP).

Furthermore, in some aspects, an internally cyclized polypeptide of the invention may be defined as a protein binding molecule, an antibiotic, an anticancer agent, an antifungal molecule or an antiviral agent. For example, in certain aspects the cyclized polypeptide may bind to a cell surface protein, such as a receptor. In some specific examples, such a cyclized polypeptide may be defined as a receptor agonist or antagonist. In still further aspects, an internally cyclized polypeptide may be defined as an antibiotic such a S. aureus antibiotic. As used herein the term antibiotic refers to a molecule that inhibits bacterial growth or pathogenesis. Thus, in some aspects a cyclic polypeptide may be a quorum-sensing polypeptide that modulates bacterial growth or gene expression. Thus, in some very specific aspects of the invention a cyclic polypeptide of the invention may be a S. aureus autoinducing polypeptide (AIP) such as AIP type I (YSTCDFIM (SEQ ID NO:1), wherein the cysteine is bonded to the C-terminal methionine via a thiolactone bridge), AIP type II, (GVNACSSLF (SEQ ID NO:2), wherein the cysteine is bonded to the C-terminal phenylalanine via a thiolactone bridge), AIP Type III (INCDFLL (SEQ ID NO:3), wherein the cysteine is bonded to the C-terminal luecine via a thiolactone bridge) or AIP Type IV (YSTCYFIM (SEQ ID NO:4), wherein the cysteine is bonded to the C-terminal methionine via a thiolactone bridge). Furthermore, in some cases quorum sensing peptide from other types of bacteria such as Enterococcus faecalis and Listeria monocytogenes may be produced by methods of the invention (Autret et al., 2003; Nakayama et al., 2001).

In still further aspects, methods of the invention may further involve a purification step. For example a method may comprise (b) purifying the fusion protein from the cell prior to exposing the fusion protein to reducing conditions. In other cases however, fusion protein such a protein secreted from a cell may be exposed to reducing conditions to generate an internally cyclized polypeptide which may subsequently be purified. As used herein the term “purified” means that the concentration of a protein (e.g., an intein fusion protein or cyclic polypeptide) is increased relative to other unrelated proteins, lipids or nucleic acids in a composition. Thus, in some cases, an intein fusion protein or a cyclic polypeptide of the invention may comprise an anchoring domain such a purification tag that may be used in the purification process. For example, a purification tag may be used to immobilize an intein fusion protein on surface such a bead or column and then the unbound molecules may be washed away. Subsequently the immobilized fusion protein may be treated with a reducing agent (e.g., a non-thiol reducing agent) thereby releasing the internally cyclized polypeptide.

In still further embodiments of the invention there is provided a method for making a plurality of distinct internally cyclized polypeptides comprising expressing a plurality of fusion proteins, wherein the fusion proteins comprise i) a functional intein polypeptide fused to the carboxy-terminus of ii) a plurality of distinct polypeptide domains having at least one internal cysteine or serine residue and (c) exposing the plurality fusion proteins to reducing conditions, thereby producing a plurality of distinct internally cyclized polypeptide and free intein polypeptide. As used herein the term distinct refers to internally cyclized polypeptides that differ in amino acid sequence at least one amino acid position. For example, a plurality of distinct polypeptide domains (having at least one internal cysteine or serine residue) may, in some cases, comprise at least one amino acid position that is randomized. In yet further cases, at least 2, 3, 4, 5, 6, 7, 8, 9, 10 or more of the amino acid positions may be randomized. In still further aspects of the invention, a plurality of distinct polypeptide domains may comprise an internal serine or cysteine residue at a constant position in the polypeptide domain. Thus, in certain instances, the amino acid(s) at positions located C-terminal relative to a cysteine or serine residue may be randomized. In this embodiment resultant internally cyclized polypeptides will comprising random amino acid sequence(s) with the cyclic domain. In certain other aspects, amino acid sequence(s) positioned N-terminally relative to the serine or cysteine residue may be randomized thereby resulting in an internally cyclized polypeptide comprising random amino acid sequence(s) in the linear domain. In still further aspects of the invention sequence located both N-terminally and C-terminally relative to the serine or cysteine position may be randomized thereby resulting in an internally cyclized polypeptide comprising random amino acids in both the linear and cyclic domains. In a very specific embodiment, for example, a functional intein polypeptide may be fused to the carboxy-terminus of an 8 amino acid polypeptide domain wherein amino acid position 4 is a cysteine or serine and amino acid positions 5 through 8 are randomized. In this case, the resultant population of internally cyclized polypeptides comprises a constant linear region of three amino acids and a lactone or thiolactone ring with 4 random amino acids. Thus, in some aspects, a plurality of distinct intein fusion proteins or distinct internally cyclized polypeptides may be defined as a library of distinct molecules. In certain aspects such a library of distinct intein fusion proteins may be defined as comprising at least about 1×10⁴, 1×10⁵, 1×10⁶, 1×10⁷, 1×10⁸, 1×10⁹, or more distinct polypeptide domains having at least one internal cysteine or serine residue. The peptides maybe expressed from an expression construct comprising an intein coding region fused to a randomly generated peptide coding region, said coding region comprising at least one fixed or floating cysteine or serine residue.

In still further aspects of the invention there is provided a method for producing an internally cyclized polypeptide having a specific biological activity. In this aspect a method of the invention may comprise producing as an internally cyclized polypeptide as described supra, (d) assessing a specific biological activity of at least a first internally cyclized polypeptide and (e) selecting at least a first internally cyclized polypeptide with a specific biological activity. Thus, in some aspects there is provided a method for screening a library for distinct internally cyclized polypeptide for a specific biological activity. As used herein the term “specific biological activity” includes but is not limited to a protein binding affinity, an anticancer activity (e.g., an antitumor activity), an antibiotic activity, antiviral activity and antifungal activity.

As used herein the specification, “a” or “an” may mean one or more. As used herein in the claim(s), when used in conjunction with the word “comprising”, the words “a” or “an” may mean one or more than one. The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more. Throughout this application, the term “about” is used to indicate that a value includes the inherent variation of error for the device, the method being employed to determine the value, or the variation that exists among the study subjects.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings are part of the present specification and are included to further demonstrate certain aspects of the present invention. The invention may be better understood by reference to the drawings in combination with the detailed description of specific embodiments presented herein.

FIG. 1—The four AIP signals of S. aureus and the cross-inhibitory groups. The amino acid sequence of each of the four AIPs is shown, and the signals are boxed into three inhibitory classes. AIP-I and AIP-IV only differ by one amino acid and function interchangeably.

FIGS. 2A-B—Schematic of intein-catalyzed protein splicing and thiolactone formation. (FIG. 2A) A simplified version of the protein splicing mechanism catalyzed by an intein. Abbreviation: X, S or O from a cysteine, serine, or threonine side chain. Details of the mechanism have been reviewed elsewhere (Perler, 2006). (FIG. 2B) The interruption of the intein splicing to generate peptide thiolactones. Following the N—S acyl shift, intramolecular attack from a cysteine side-chain generates the thiolactone ring.

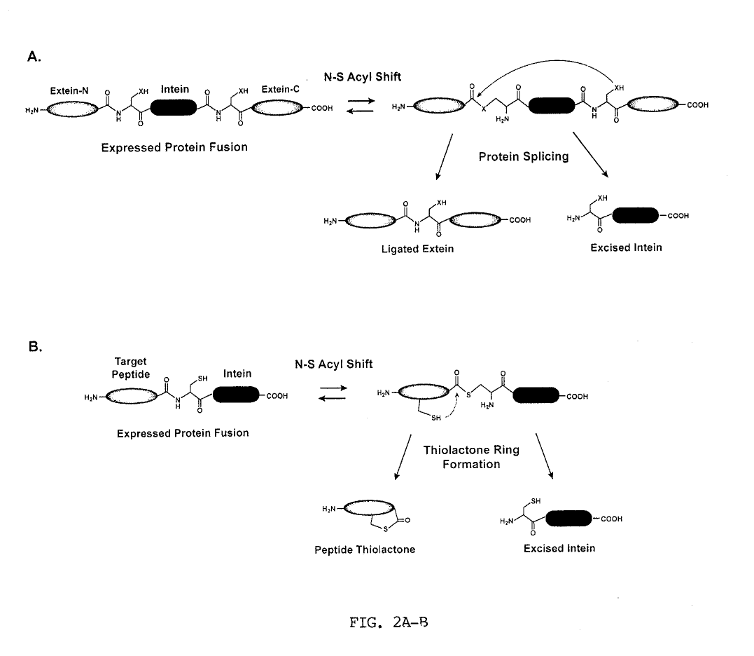

FIG. 3—Schematic of the method for generating the S. aureus AIP signals using the DnaB mini-intein. Firstly, an oligonucleotide encoding the AIP peptide is ligated at the 5′ end of the DnaB intein in plasmid pDnaB8. The construct is expressed in E. coli, cells are lysed, and the fusion protein is purified on resin (CBD=chitin-binding domain). The intein performs the N—S acyl shift creating the thioester, allowing internal attack from a cysteine side-chain to release the thiolactone-containing peptide. Elution fractions are then tested for biological activity with S. aureus reporter strains.

FIGS. 4A-B—DnaB intein activity and AIP-I purification. (FIG. 4A) A dihydrofolate reductase (DHFR) protein fusion was used to test DnaB activity. Plasmids pDnaB8, pDnaB8-DHFR, and pET22-bsDHFR were expressed in strain AH394 with or without IPTG induction as indicated. Overexpressed bands corresponding to the intein-CBD (chitin binding domain), DHFR-intein-CBD, and DHFR are as shown. (FIG. 4B) Samples of an iAIP-I purification were separated by SDS-PAGE and probed with CBD antibody (shown on top). Gel lanes are as follows: SM, size marker; Un, uninduced; Ind, induced; FT-1, early flow through sample; FT-2, late flow through sample; Resin, chitin resin.

FIG. 5—Verification of the iAIP-I structure. Strain AH430 (Agr-II) served as the reporter for all the tests, and GFP readings were taken 12 hr after sample addition. For testing, each sample was diluted 20-fold into the AH430 culture at the beginning of logarithmic phase. As controls, TSB and supernatant from SH1000 (AIP-I) were added to AH430. To test the DnaB intein method, iAIP-I was purified from strains AH426 (shown as iAIP-I) and AH425 (cysteine mutant, shown as Mut). As indicated, the samples were left untreated, and to check for the thiolactone ring, the samples were treated with base or hydroxylamine.

FIG. 6—Inhibition profiling with the iAIPs. For testing, each sample was diluted 20-fold into the appropriate reporter strain at the beginning of log phase. GFP fluorescence was monitored over time and compared to control samples of TSB and filtered supernatants from AIP-I, AIP-II, and AIP-III producing strains. (FIG. 6A) Strain AH429 (Agr-I reporter). (FIG. 6B) AH430 (Agr-II reporter). (FIG. 6C) Strain AH431 (Agr-III reporter).

FIG. 7.—Agr activation with the iAIPs. S. aureus Agr-I (AH462), Agr-II (AH430), Agr-III (AH431) reporter strains were grown in TSB with 0.2% glucose, and 50 nM iAIP was added at the beginning of logarithmic phase. Over time, GFP fluorescence was monitored and compared to controls without additions (TSB) or with competing iAIP signals. Activation or inhibition results with control S. aureus supernatants are not shown, but all yielded the same pattern as observed with the iAIPs.

### DETAILED DESCRIPTION OF THE INVENTION

Internally cyclized peptides and polypeptides are known to have a variety of important biological functions. For example, thiolactone peptides such as Staphylococcal AIPs are involved in bacterium quorum sensing and may be used to regulation bacterial growth an pathogenesis. However, difficulties in the chemical synthesis of cyclic peptides have limited their study and implementation as therapeutics. Furthermore, the sterically constrained ring of cyclic polypeptides results in molecules that may be ideal candidates for pathway specific agonism or antagonism. However, the cost and difficulty in chemical synthesis of peptides constrained by lactone and thiolactone rings has also limited the ability build and screen libraries of cyclic polypeptides for such activity.

The present invention addresses the deficiencies in the prior art by providing a biological platform for efficiently synthesizing internally cyclized polypeptides and for making a library of cyclic polypeptides that may be screened for biological activity. Studies presented herein demonstrate that peptides (such as S. aureus AIP type I) may be fused with intein proteins and expressed in cells. These fusion proteins are purified from the cells and by virtue of the intein catalytic activity will form an internally cyclized polypeptide upon exposure to reducing conditions. Specifically, the functional intein polypeptide catalyzes an N→S acyl shift to generate a thioester bond in the fusion protein at the amino terminus of the intein polypeptide. Next, under reducing conditions, an internal serine, threonine or cysteine residue within fused peptide attacks the activated carbon in the thioester to form a thiolactone (or lactone) bridge. Thus, when complete, the reaction severs the covalent bond between the polypeptide domain and the intein domain to release a free intein domain and an internally cyclized polypeptide comprising either a lactone or thiolactone bridge. Cyclic AIP type I peptides made by this method are active demonstrating that the new methods may be used to efficiently produce active internally cyclized peptides.

Thus, the studies herein provide a new method for biologically producing a cyclic peptide of interest. Furthermore, these new genetic production methods provide a platform for generating cyclic peptide libraries. For instance randomized or partially randomized sequence may be fused to an active intein and expressed in cells. Cells comprising individual clones are grown out and the fusion proteins are purified and induced to form lactone or thiolactone bridges by exposure to a reducing agent. The resultant cyclic polypeptides may then be screened for a biologic activity, such as an antibiotic activity. Molecules that are found to be active may easily identified by cloning the indicated expression cassette from bacteria. Thus, the invention provided high through-put methods for identifying new biologically active cyclic polypeptides.

### I. NUCLEIC ACIDS

The present invention concerns a number of different types of nucleic acid molecules that can be used in a variety of ways. For example, nucleic acids may comprise an expression cassette comprising an intein fusion protein of the invention. In some embodiments of the invention, the nucleic acid is a recombinant nucleic acid. The term “recombinant” is used according to its ordinary and plain meaning to refer to the product of recombinant DNA technology, e.g., genetically engineered DNA prepared in vitro by cutting up DNA molecules and splicing together specific DNA fragments, which may or may not be from different organisms. Things that have or are from a genetically engineered DNA are similarly recombinant; this includes replicated or duplicated products based on the initially engineered DNA. In particular embodiments, the invention concerns therapeutic nucleic acids recombinant DNA and RNA molecules. In some embodiments, the nucleic acid molecule is a DNA molecule, for example, the DNA molecule may be used in to express a fusion protein of the invention.

Embodiments of the invention concern isolated and/or recombinant polynucleotides. An isolated polynucleotide refers to a polynucleotide that is separated from a cell and its non-nucleic acid contents, and more specifically, may be separated from other nucleic acid sequences. A recombinant polynucleotide refers to a genetically engineered nucleic acid molecule or products of such a molecule (either through duplication, replication, or expression).

The term “cDNA” is intended to refer to DNA prepared using messenger RNA (mRNA) as template. In many embodiments of the invention, the nucleic acid is a cDNA or cDNA sequence. For example, DNA sequences encoding an intein fusion protein may be defined as cDNA. The advantage of using a cDNA, as opposed to genomic DNA or DNA polymerized from a genomic, non- or partially-processed RNA template, is that the cDNA primarily contains coding sequences of the corresponding protein. There may be times when the full or partial genomic sequence is preferred, such as where the non-coding regions are required for optimal expression (e.g., to avoid non-sense mediated decay (NMD) in eukaryotic cells).

In other embodiments, the invention concerns isolated nucleic acid molecules and recombinant vectors incorporating DNA sequences that encode a polypeptide or peptide that includes within its amino acid sequence a contiguous amino acid sequence in accordance with, or essentially corresponding to the polypeptide.

The nucleic acid segments used in the present invention, regardless of the length of the coding sequence itself, may be combined with other nucleic acid sequences, such as promoters, polyadenylation signals, additional restriction enzyme sites, multiple cloning sites, other coding segments, and the like, such that their overall length may vary considerably. It is therefore contemplated that a nucleic acid fragment of almost any length may be employed, with the total length preferably being limited by the ease of preparation and use in the intended recombinant DNA protocol.

It is contemplated that the nucleic acid constructs of the present invention may encode part or all (full-length) of transcripts or polypeptides from any source. Alternatively, a nucleic acid sequence may encode an RNA or polypeptide with additional heterologous sequences, for example to allow for purification of the polypeptide, transport, secretion, post-translational modification, or for therapeutic benefits such as targeting or efficacy. As discussed above, a tag or other heterologous polypeptide may be added to the intein fusion protein-encoding sequence, wherein “heterologous” refers to a sequence that is not the same from the same source as other sequences.

In certain other embodiments, the invention concerns isolated DNA or RNA segments and recombinant vectors that include within their sequence the coding sequence for an intein fusion protein. One of skill in the art will understand the due to the degeneracy of the genetic code a variety of nucleic acid sequence can encode a single amino acid sequence (see for instance the codons listed in Table 1). Therefore, it is contemplated that any nucleic acid sequence capable of encoding a polypeptide of the invention is included as part of the instant invention.

TABLE 1
Examplary DNA Codons

| Amino Acids | | | Codons |
|---|---|---|---|
| Alanine | Ala | A | GCC GCT GCA GCG |
| Cysteine | Cys | C | TGC TGT |
| Aspartic acid | Asp | D | GAC GAT |
| Glutamic acid | Glu | E | GAG GAA |
| Phenylalanine | Phe | F | TTC TTT |
| Glycine | Gly | G | GGC GGG GGA GGT |
| Histidine | His | H | CAC CAT |
| Isoleucine | Ile | I | ATC ATT ATA |
| Lysine | Lys | K | AAG AAA |
| Leucine | Leu | L | CTG CTC TTG CTT CTA TTA |
| Methionine | Met | M | ATG |
| Asparagine | Asn | N | AAC AAT |
| Proline | Pro | P | CCC CCT CCA CCG |
| Glutamine | Gln | Q | CAG CAA |
| Arginine | Arg | R | CGC AGG CGG AGA CGA CGT |
| Serine | Ser | S | AGC TCC TCT AGT TCA TCG |
| Threonine | Thr | T | ACC ACA ACT ACG |
| Valine | Val | V | GTG GTC GTT GTA |
| Tryptophan | Trp | W | TGG |
| Tyrosine | Tyr | Y | TAC TAT |

A number of additional embodiments in the context of nucleic acids are discussed below.

A. Vectors

In some aspects, peptides and/or polypeptides of the invention may be encoded by a nucleic acid molecule comprised in a vector. The term “vector” is used to refer to a carrier nucleic acid molecule into which a nucleic acid sequence can be inserted for introduction into a cell where it can be replicated. A nucleic acid sequence can be “exogenous,” which means that it is foreign to the cell into which the vector is being introduced or that the sequence is homologous to a sequence in the cell but in a position within the host cell nucleic acid in which the sequence is ordinarily not found. Vectors include plasmids, cosmids, viruses (bacteriophage, animal viruses, and plant viruses), and artificial chromosomes (e.g., YACs). One of skill in the art would be well equipped to construct a vector through standard recombinant techniques, which are described in Sambrook et al., (1989) and Ausubel et al., 1996, both incorporated herein by reference. A targeting molecule is one that directs the modified polypeptide to a particular organ, tissue, cell, or other location in a subject's body.

The term “expression vector” refers to a vector containing a nucleic acid sequence coding for at least part of a gene product capable of being transcribed. In some cases, RNA molecules are then translated into a protein, polypeptide, or peptide. In other cases, these sequences are not translated, for example, in the production of RNA molecules used in methods of the invention. Expression vectors can contain a variety of “control sequences,” which refer to nucleic acid sequences necessary for the transcription and possibly translation of an operably linked coding sequence in a particular host organism. For instance, in some embodiments of the invention, there may sequences to allow for in vitro transcription of a sequence. In particular embodiments, the expression vector may contain an Sp6, T3, or T7 promoter. In addition to control sequences that govern transcription and translation, vectors and expression vectors may contain nucleic acid sequences that serve other functions as well and are described infra.

A “promoter” is a control sequence that is a region of a nucleic acid sequence at which initiation and rate of transcription are controlled. It may contain genetic elements at which regulatory proteins and molecules may bind such as RNA polymerase and other transcription factors. The phrases “operatively positioned,” “operatively linked,” “under control,” and “under transcriptional control” mean that a promoter is in a correct functional location and/or orientation in relation to a nucleic acid sequence to control transcriptional initiation and/or expression of that sequence. A promoter may or may not be used in conjunction with an “enhancer,” which refers to a cis-acting regulatory sequence involved in the transcriptional activation of a nucleic acid sequence.

A promoter may be one naturally associated with a gene or sequence, as may be obtained by isolating the 5′ non-coding sequences located upstream of the coding segment and/or exon. Such a promoter can be referred to as “endogenous.” Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter, which refers to a promoter that is not normally associated with a nucleic acid sequence in its natural environment. A recombinant or heterologous enhancer refers also to an enhancer not normally associated with a nucleic acid sequence in its natural environment. Such promoters or enhancers may include promoters or enhancers of other genes, and promoters or enhancers isolated from any other prokaryotic, viral, or eukaryotic cell, and promoters or enhancers not “naturally occurring,” e.g., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR™, in connection with the compositions disclosed herein (see U.S. Pat. No. 4,683,202; U.S. Pat. No. 5,928,906, each incorporated herein by reference). Furthermore, it is contemplated the control sequences that direct transcription and/or expression of sequences within non-nuclear organelles such as mitochondria, chloroplasts, and the like, can be employed as well.

Naturally, it may be important to employ a promoter and/or enhancer that effectively directs the expression of the DNA segment in the cell type, organelle, and organism chosen for expression. Those of skill in the art of molecular biology generally know the use of promoters, enhancers, and cell type combinations for protein expression, for example, see Sambrook et al. (1989), incorporated herein by reference. The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous.

In certain embodiments of the invention, a vector may also include one or more of: an ATG initiation signal, internal ribosome binding sites, multiple cloning site (MCS), splicing site, termination signal, polyadenylation signal, origin of replication, or selectable or screenable marker (drug resistance marker, enzymatic marker, colorimetric marker, fluorescent marker).

B. Host Cells

As used herein, the terms “cell,” “cell line,” and “cell culture” may be used interchangeably. All of these terms also include their progeny, which is any and all subsequent generations. It is understood that all progeny may not be identical due to deliberate or inadvertent mutations. In the context of expressing a heterologous nucleic acid sequence, “host cell” refers to a prokaryotic or eukaryotic cell, and it includes any transformable organisms that is capable of replicating a vector and/or expressing a heterologous gene encoded by a vector. Such a host cell would be considered recombinant if the heterologous nucleic acid sequence was the product of recombinant DNA technology. A host cell can, and has been, used as a recipient for vectors. A host cell may be “transfected” or “transformed,” which refers to a process by which exogenous nucleic acid, such as a modified protein-encoding sequence, is transferred or introduced into the host cell. A transformed cell includes the primary subject cell and its progeny.

Host cells for use according to the invention may be derived from prokaryotes such as bacteria or eukaryotes, including yeast cells, insect cells, and mammalian cells, depending upon whether the desired result is replication of the vector or expression of part or all of the vector-encoded nucleic acid sequences. In certain embodiments, the cell is an embryonic stem cell, such as from a mouse.

Numerous cell lines and cultures are available for use as a host cell, and they can be obtained through the American Type Culture Collection (ATCC), which is an organization that serves as an archive for living cultures and genetic materials (World Wide Web at atcc.org). An appropriate host can be determined by one of skill in the art based on the vector backbone and the desired result. A plasmid or cosmid, for example, can be introduced into a prokaryote host cell for replication of many vectors. Bacterial cells used as host cells for vector replication and/or expression include but are not limited to DH5α, JM109, and KC8, as well as a number of commercially available bacterial hosts such as SURE® Competent Cells and SOLOPACK™ Gold Cells (STRATAGENE®, La Jolla, Calif.). Alternatively, bacterial cells such as E. coli LE392 could be used as host cells for phage viruses. Appropriate yeast cells include Saccharomyces cerevisiae, Saccharomyces pombe, and Pichia pastoris.

Examples of eukaryotic host cells for replication and/or expression of a vector include HeLa, NIH3T3, Jurkat, 293, Cos, CHO, Saos, SF9, and PC12. Many host cells from various cell types and organisms are available and would be known to one of skill in the art. Similarly, a viral vector may be used in conjunction with either a eukaryotic or prokaryotic host cell, particularly one that is permissive for replication or expression of the vector.

Some vectors may employ control sequences that allow it to be replicated and/or expressed in both prokaryotic and eukaryotic cells. One of skill in the art would further understand the conditions under which to incubate all of the above described host cells to maintain them and to permit replication of a vector. Also understood and known are techniques and conditions that would allow large-scale production of vectors, as well as production of the nucleic acids encoded by vectors and their cognate polypeptides, proteins, or peptides.

C. Expression Systems

Numerous expression systems exist that comprise at least a part or all of the compositions discussed above. Prokaryote- and/or eukaryote-based systems can be employed for use with the present invention to produce nucleic acid sequences, or their cognate polypeptides, proteins and peptides. For example, high yield expression in sect cells such as SF-9 cells, may be accomplished by baculoviral expression systems. Another useful eukaryotic expression system is yeast which can be used to produce relatively large amounts of protein at a low cost. Many such systems are commercially and widely available.

D. Peptide/Polypeptide-Encoding Libraries

In certain aspects of the invention, one will provide libraries of nucleic acids containing distinct sequence that encode for distinct peptides/polypeptides. For example, it will be useful to generate random or pseudo-random cysteine/serine-containing peptides by randomly synthesizing oligo- or polynucleotides that contain at least one cysteine/serine encoding codon, which can be located in a fixed (pseudo-random) or “floating” (random) position. Techniques for generating such oligo- or polynucleotides are well known in the art.

### II. PROTEINACEOUS COMPOSITIONS

In certain embodiments, the present invention concerns compositions comprising at least one proteinaceous molecule, such as an intein fusion protein or internally cyclized polypeptide. As used herein, a “proteinaceous molecule,” “proteinaceous composition,” “proteinaceous compound,” “proteinaceous chain” or “proteinaceous material” generally refers, but is not limited to, a protein molecule containing at least one polypeptide with multiple amino acids. The protein may contain more than one polypeptide, such as a dimer or trimer or other tertiary structure. In some embodiments, a protein refers to a polypeptide that has 3 amino acids or more or to a peptide of from 3 to 100 amino acids. All the “proteinaceous” terms described above may be used interchangeably herein. In the case of a protein composed of a single polypeptide, the terms “polypeptide” and “protein” are used interchangeably.

In certain embodiments the size of the at least one proteinaceous molecule may comprise, or be at most or at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600 or greater amino molecule residues, and any range derivable therein. Moreover, it may contain such lengths of contiguous amino acids from a polypeptide provided herein, such as an intein.

As used herein, an “amino molecule” refers to any amino acid, amino acid derivative or amino acid mimic as would be known to one of ordinary skill in the art. In certain embodiments, the residues of the proteinaceous molecule are sequential, without any non-amino molecule interrupting the sequence of amino molecule residues. In other embodiments, the sequence may comprise one or more non-amino molecule moieties. In particular embodiments, the sequence of residues of the proteinaceous molecule may be interrupted by one or more non-amino molecule moieties.

Accordingly, the term “proteinaceous composition” encompasses amino molecule sequences comprising at least one of the 20 common amino acids in naturally synthesized proteins.

In certain embodiments the proteinaceous composition comprises at least one protein, polypeptide or peptide. In further embodiments the proteinaceous composition comprises a biocompatible protein, polypeptide or peptide. As used herein, the term “biocompatible” refers to a substance which produces no significant untoward effects when applied to, or administered to, a given organism according to the methods and amounts described herein. Such untoward or undesirable effects are those such as significant toxicity or adverse immunological reactions. In preferred embodiments, biocompatible protein, polypeptide or peptide containing compositions will generally be proteins or peptides or synthetic proteins or peptides each essentially free from toxins, pathogens and harmful immunogens.

Proteinaceous compositions may be made by any technique known to those of skill in the art, including the expression of proteins, polypeptides or peptides through standard molecular biological techniques and as described supra. The nucleotide and protein, polypeptide and peptide sequences for various genes have been previously disclosed, and may be found at computerized databases known to those of ordinary skill in the art. One such database is the National Center for Biotechnology Information's Genbank and GenPept databases (on the World Wide Web at ncbi.nlm.nih.gov/). The coding regions for these known genes may be amplified and/or expressed using the techniques disclosed herein or as would be know to those of ordinary skill in the art. Alternatively, various commercial preparations of proteins, polypeptides and peptides are known to those of skill in the art.

In certain embodiments and as described supra a proteinaceous compound may be purified. Generally, “purified” will refer to a specific or protein, polypeptide, or peptide composition that has been subjected to fractionation to remove various other proteins, polypeptides, or peptides, and which composition substantially retains its activity, as may be assessed, for example, by the protein assays, as would be known to one of ordinary skill in the art for the specific or desired protein, polypeptide or peptide.

In additional aspects of the invention polypeptide sequences may be further modified by amino substitutions, for example by substituting an amino acid at one or more positions with an amino acid having a similar hydrophilicity. The importance of the hydropathic amino acid index in conferring interactive biologic function on a protein is generally understood in the art (Kyte & Doolittle, 1982). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like. Thus such conservative substitution can be made in polypeptides of the invention and will likely only have minor effects on their biological activity. As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (0.5); histidine −0.5); cysteine (−1.0); methionine (−1.3); valine (−1.5); leucine (−1.8); isoleucine (−1.8); tyrosine (2.3); phenylalanine (−2.5); tryptophan (−3.4). These values can be used as a guide and thus substitution of amino acids whose hydrophilicity values are within ±2 are preferred, those that are within ±1 are particularly preferred, and those within ±0.5 are even more particularly preferred. Thus, any of the polypeptides described herein may be modified by the substitution of an amino acid, for different, but homologous amino acid with a similar hydrophilicity value. Amino acids with hydrophilicities within +/−1.0, or +/−0.5 points are considered homologous. Some examples of homologous amino acids substitutions listed in Table 2 below.

TABLE 2
Exemplary Amino Acid Aubstitutions

| Original Residue | Exemplary Substitutions |
|---|---|
| Ala | Gly; Ser |
| Arg | Lys |
| Asn | Gln; His |
| Asp | Glu |
| Cys | Ser |
| Gln | Asn |
| Glu | Asp |
| Gly | Ala |
| His | Asn; Gln |
| Ile | Leu; Val |
| Leu | Ile; Val |
| Lys | Arg |
| Met | Met; Leu; Tyr |
| Ser | Thr |
| Thr | Ser |
| Trp | Tyr |
| Tyr | Trp; Phe |
| Val | Ile; Leu |

Accordingly, sequences that have between about 70% and about 80%, between about 81% and about 90%; or between about 91% and about 99%; of amino acids that are identical or functionally equivalent to the amino acids of a reference polypeptide sequence are included as part of the invention.

A specialized kind of insertional variant is the fusion protein. This molecule generally has all or a substantial portion of the native molecule, linked at the N- or C-terminus, to all or a portion of a second polypeptide. For example, fusions typically employ leader sequences from other species to permit the recombinant expression of a protein in a heterologous host. Another useful fusion includes the addition of an immunologically active domain, such as an antibody epitope, to facilitate purification of the fusion protein. Inclusion of a cleavage site at or near the fusion junction will facilitate removal of the extraneous polypeptide after purification. Other useful fusions include linking of functional domains, such as active sites from enzymes such as a hydrolase, glycosylation domains, cellular targeting signals or transmembrane regions.

A. Protein Purification

In some embodiments, it may be desirable to purify a protein, for example, an intein fusion protein or cyclic polypeptide. Protein purification techniques are well known to those of skill in the art. These techniques involve, at one level, the crude fractionation of the cellular milieu to polypeptide and non-polypeptide fractions. Having separated the polypeptide from other proteins, the polypeptide of interest may be further purified using chromatographic and electrophoretic techniques to achieve partial or complete purification (or purification to homogeneity). Analytical methods particularly suited to the preparation of a pure peptide or polypeptide are filtration, ion-exchange chromatography, exclusion chromatography, polyacrylamide gel electrophoresis, affinity chromatography, or isoelectric focusing. A particularly efficient method of purifying peptides is fast protein liquid chromatography or even HPLC.

Certain aspects of the present invention concern the purification, and in particular embodiments, the substantial purification, of an encoded protein or peptide. The term “purified protein or peptide” as used herein, is intended to refer to a composition, isolatable from other components, wherein the protein or peptide is purified to any degree relative to its naturally-obtainable state. A purified protein or peptide therefore also refers to a protein or peptide, free from the environment in which it may naturally occur.

Generally, “purified” will refer to a protein or peptide composition that has been subjected to fractionation to remove various other components, and which composition substantially retains its expressed biological activity. Where the term “substantially purified” is used, this designation will refer to a composition in which the protein or peptide forms the major component of the composition, such as constituting about 50%, about 60%, about 70%, about 80%, about 90%, about 95% or more of the proteins in the composition.

Various methods for quantifying the degree of purification of the protein or peptide will be known to those of skill in the art in light of the present disclosure. These include, for example, determining the specific activity of an active fraction, or assessing the amount of polypeptides within a fraction by SDS/PAGE analysis. A preferred method for assessing the purity of a fraction is to calculate the specific activity of the fraction, to compare it to the specific activity of the initial extract, and to thus calculate the degree of purity, herein assessed by a “-fold purification number.” The actual units used to represent the amount of activity will, of course, be dependent upon the particular assay technique chosen to follow the purification and whether or not the expressed protein or peptide exhibits a detectable activity.

Various techniques suitable for use in protein purification will be well known to those of skill in the art. These include, for example, precipitation with ammonium sulfate, PEG, antibodies and the like or by heat denaturation, followed by centrifugation; chromatography steps such as ion exchange, gel filtration, reverse phase, hydroxylapatite and affinity chromatography; isoelectric focusing; gel electrophoresis; and combinations of such and other techniques. As is generally known in the art, it is believed that the order of conducting the various purification steps may be changed, or that certain steps may be omitted, and still result in a suitable method for the preparation of a substantially purified protein or peptide.

There is no general requirement that the protein or peptide always be provided in their most purified state. Indeed, it is contemplated that less substantially purified products will have utility in certain embodiments. Partial purification may be accomplished by using fewer purification steps in combination, or by utilizing different forms of the same general purification scheme. For example, it is appreciated that a cation-exchange column chromatography performed utilizing an HPLC apparatus will generally result in a greater “-fold” purification than the same technique utilizing a low pressure chromatography system. Methods exhibiting a lower degree of relative purification may have advantages in total recovery of protein product, or in maintaining the activity of an expressed protein.

It is known that the migration of a polypeptide can vary, sometimes significantly, with different conditions of SDS/PAGE (Capaldi et al., 1977). It will therefore be appreciated that under differing electrophoresis conditions, the apparent molecular weights of purified or partially purified expression products may vary.

B. Antibodies

Another embodiment of the present invention may involve antibodies. In some cases, for example an antibody may be used to purify or detect an intein fusion protein or a cyclized polypeptide.

mAbs may be readily prepared through use of well-known techniques, such as those exemplified in U.S. Pat. No. 4,196,265, incorporated herein by reference. Typically, this technique involves immunizing a suitable animal with a selected immunogen composition, e.g., a purified or partially purified polypeptide, peptide or domain, be it a wild-type or mutant composition. The immunizing composition is administered in a manner effective to stimulate antibody producing cells.

Antibodies may be further purified, if desired, using filtration, centrifugation and various chromatographic methods such as HPLC or affinity chromatography. Fragments of the monoclonal antibodies of the invention can be obtained from the monoclonal antibodies so produced by methods which include digestion with enzymes, such as pepsin or papain, and/or by cleavage of disulfide bonds by chemical reduction. Alternatively, antibody fragments encompassed by the present invention can be synthesized using an automated peptide synthesizer. It also is contemplated that a molecular cloning approach may be used to generate antibodies.

C. Inteins

Inteins have been identified in every branch of organisms and a database of known inteins has recently been constructed (Perler, 2000; Perler, 2002; www.neb.com/neb/inteins.html). Any catalytically active intein may be used in the methods of the invention. For example in some cases an intein for use in the invention maybe a eukaryotic intein such as the APMV Pol, Abr PRP8, Aca PRP8, Afu PRP8, Ani PRP8, Avi PRP8, Bci PRP8, Bde-JEL197 RPB2, Bde-JEL423 PRP8-1, Bde-JEL423 PRP8-2, Bde-JEL423 RPC2, Bde-JEL423 eIF-5B, CIV RIR1, CV-NY2A ORF212392, CV-NY2A RIR1, CZIV RIR1, Cba PRP8, Ceu C1pP, Cga PRP8, Cgl VMA, Cla PRP8, Cne-A PRP8 (Fne-A PRP8), Cne-AD PRP8 (Fne-AD PRP8), Cne-JEC21 PRP8, Cpa ThrRS, Cre RPB2, Cst RPB2, Ctr ThrRS, Ctr VMA, Ddi RPC2, Dhan GLT1, Dhan VMA, Eni PRP8, Gth DnaB, HaV01 Pol, Hca PRP8, Kla-CBS683 VMA, Kla-IFO1267 VMA, Kpo VMA, Le1 VMA, Nfi PRP8, Ng1-FR2163 PRP8, Ng1-FRR1833 PRP8, Nqu PRP8, Nspi PRP8, Pan CHS2, Pan GLT1, Pbr PRP8, Pch PRP8, Pex PRP8, Pgu GLT1, Pgu-alt GLT1, Pno GLT1, Pno RPA2, Ppu DnaB, Pst VMA, Pvu PRP8, Pye DnaB, Sas RPB2, Sca VMA, Scar VMA, Sce VMA, Sce-DH1-1A VMA, Sce-OUT7091 VMA, Sce-OUT7112 VMA, Sda VMA, Sex VMA-1, Sex VMA-2, She RPB2 (RpoB), Sja VMA, Spa VMA, Spu PRP8, Sun VMA, Tg1 VMA, Tpr VMA, Ure PRP8, WIV RIR1, Zba VMA, Zbi VMA or the Zro VMA intein or a catalytically active fragment thereof.

In certain other aspects, an intein for use in the invention may be defined as a bacterial intein as the AP-APSE1 dpol, Aae RIR2, Ace RIR1, Aeh DnaB-1, Aeh DnaB-2, Aeh RIR1, Aha DnaE-c, Aha DnaE-n, Aov DnaE-c, Aov DnaE-n, Arsp-FB24 DnaB, Asp DnaE-c, Asp DnaE-n, Ava DnaE-c, Ava DnaE-n, Avin RIR1 BIL, Bee DnaB, BsuP-M1918 RIR1, BsuP-SPBc2 RIR1, CP-Cth TerA, Cag RIR1, Cau SpoVR, Cbu DnaB, Cch RIR1, Chy RIR1, Cth ATPase BIL, Cwa DnaB, Cwa DnaE-c, Cwa DnaE-n, Cwa PEP, Cwa RIR1, Dge DnaB, Dha-DCB2 RIR1, Dha-Y51 RIR1, Dra RIR1, Dra Snf2-c, Dra Snf2-n, Dra-ATCC13939 Snf2, Fal DnaB, Fsp-CcI3 RIR1, Gob DnaE, Gob Hyp, Gvi DnaB, Gvi RIR1-1, Gvi RIR1-2, Kra DnaB, LP-phiHSIC Helicase, MP-Aaphi23 MupF, MP-Be DnaB, MP-Be gp51, MP-Catera gp206, MP-Mcjw1 DnaB, MP-Omega DnaB, MP-U2 gp50, Mav DnaB, Mav-PT DnaB, Mbo DnaB, Mbo Pps1, Mbo RecA, Mbo SufB (Mbo Pps1), Mca MupF, Mca RIR1, Mch RecA, Mex Helicase, Mex TrbC, Mfa RecA, Mfl GyrA, Mfl RecA, Mfl-ATCC14474 RecA, Mf1-PYR-GCK DnaB, Mga GyrA, Mga RecA, Mga SufB (Mga Pps1), Mgo GyrA, Min DnaB, Mkas GyrA, Mle DnaB, Mle GyrA, Mle RecA, Mle SufB (Mle Pps1), Mma GyrA, Mmag Magn8951 BIL, Msh RecA, Msm DnaB-1, Msm DnaB-2, Msp-KMS DnaB, Msp-KMS GyrA, Msp-MCS DnaB, Msp-MCS GyrA, Mthe RecA, Mtu SufB (Mtu Pps1), Mtu-CDC1551 DnaB, Mtu-H37Rv DnaB, Mtu-H37Rv RecA, Mtu-So93 RecA, Mva DnaB, Mxa RAD25, Mxe GyrA, Nfa DnaB, Nfa Nfa15250, Nfa RIR1, Npu DnaB, Npu DnaE-c, Npu DnaE-n, Npu GyrB, Nsp-JS614 DnaB, Nsp-JS614 TOPRIM, Nsp-PCC7120 DnaB, Nsp-PCC7120 DnaE-c, Nsp-PCC7120 DnaE-n, Nsp-PCC7120 RIR1, Oli DnaE-c, Oli DnaE-n, PP-PhiEL Helicase, PP-PhiEL ORF11, PP-PhiEL ORF39, PP-PhiEL ORF40, Pf1 Fha BIL, Plut RIR1, Pna RIR1, Posp-JS666 DnaB, Posp-JS666 RIR1, Pssp-A1-1 Fha, Psy Fha, Rma DnaB, Rsp RIR1, SP-Sfv-2a Primase, SP-Sfv-5 Primase, SP-Twort ORF6, Sav Helicase, Sel-PC6301 RIR1, Sel-PC7942 DnaE-c, Sel-PC7942 DnaE-n, Sel-PC7942 RIR1, Sel-PCC6301 DnaE-c, Sel-PCC6301 DnaE-n, Sep RIR1, Sp1 DnaX, Sru DnaB, Sru PolBc, Ssp DnaB, Ssp DnaE-c, Ssp DnaE-n, Ssp DnaX, Ssp GyrB, Ssp-JA2 DnaB, Ssp-JA2 RIR1, Ssp-JA3 DnaB, Ssp-JA3 RIR1, Tel DnaE-c, Tel DnaE-n, Ter DnaB-1, Ter DnaB-2, Ter DnaE-1, Ter DnaE-2, Ter DnaE-3c, Ter DnaE-3n, Ter GyrB, Ter Ndse-1, Ter Ndse-2, Ter RIR1-1, Ter RIR1-2, Ter RIR1-3, Ter RIR1-4, Ter Snf2, Ter ThyX, Tfus Hyp-2914, Tfus RecA-1, Tfus RecA-2, Tth-HB27 DnaE-1, Tth-HB27 DnaE-2, Tth-HB27 RIR1-1, Tth-HB27 RIR1-2, Tth-HB8 DnaE-1, Tth-HB8 DnaE-2, Tth-HB8 RIR1-1, Tth-HB8 RIR1-2, Tvu DnaE-c or the Tvu DnaE-n intein or a catalytically active fragment thereof.

In still other aspects, an intein for use according to the invention may be an archaeal intein such as the Ape APE0745, Fac RIR1, Fac SufB (Fac Pps1), Hma CDC21, Hma Pol-II, Hma PolB, Hma TopA, Hsa-NRC1 CDC21, Hsa-NRC1 Pol-II, Hvo PolB, Hwa GyrB, Hwa Mcm-1, Hwa Mcm-2, Hwa Mcm-3, Hwa Mcm-4, Hwa Pol-II-1, Hwa Pol-II-2, Hwa PolB-1, Hwa PolB-2, Hwa RIR1-1, Hwa RIR1-2, Hwa Top6B, Hwa rPol A″, Mhu Pol-II, Mja GF-6P, Mja Helicase, Mja Hyp-1, Mja IF2, Mja K1bA, Mja PEP, Mja Pol-1, Mja Pol-2, Mja RFC-1, Mja RFC-2, Mja RFC-3, Mja RNR-1, Mja RNR-2, Mja RtcB (Mja Hyp-2), Mja TFIIB, Mja UDP GD, Mja r-Gyr, Mja rPol A′, Mja rPol A″, Mka CDC48, Mka EF2, Mka RFC, Mka RtcB, Mka VatB, Mth RIR1, Neq Pol-c, Neq Pol-n, Nph CDC21, Nph PolB-1, Nph PolB-2, Nph rPol A″, Pab CDC21-1, Pab CDC21-2, Pab IF2, Pab K1bA, Pab Lon, Pab Moaa, Pab Pol-II, Pab RFC-1, Pab RFC-2, Pab RIR1-1, Pab RIR1-2, Pab RIR1-3, Pab RtcB (Pab Hyp-2), Pab VMA, Pfu CDC21, Pfu IF2, Pfu K1bA, Pfu Lon, Pfu RFC, Pfu RIR1-1, Pfu RIR1-2, Pfu RtcB (Pfu Hyp-2), Pfu TopA, Pfu VMA, Pho CDC21-1, Pho CDC21-2, Pho IF2, Pho K1bA, Pho LHR, Pho Lon, Pho Pol I, Pho Pol-II, Pho RFC, Pho RIR1, Pho RadA, Pho RtcB (Pho Hyp-2), Pho VMA, Pho r-Gyr, Psp-GBD Pol, Pto VMA, Tac-ATCC25905 VMA, Tac-DSM1728 VMA, Tag Pol-1 (Tsp-TY Pol-1), Tag Pol-2 (Tsp-TY Pol-2), Tag Pol-3 (Tsp-TY Pol-3), Tfu Pol-1, Tfu Pol-2, Thy Pol-1, Thy Pol-2, Tko CDC21-1, Tko CDC21-2, Tko Helicase, Tko IF2, Tko K1bA, Tko LHR, Tko Pol-1 (Pko Pol-1), Tko Pol-2 (Pko Pol-2), Tko Pol-II, Tko RFC, Tko RIR1-1, Tko RIR1-2, Tko RadA, Tko TopA, Tko r-Gyr, Tli Pol-1, Tli Pol-2, Tpe Pol, Tsp-GE8 Pol-1, Tsp-GE8 Pol-2, Tsp-GT Pol-1, Tsp-GT Pol-2, Tsp-NA1 Pol, Tthi Pol, Tvo VMA, Tzi Pol, Unc-ERS PFL, Unc-ERS RIR1, Unc-ERS RNR, or the Unc-MetRFS MCM2 intein or a catalytically active fragment thereof.

### III. SCREENING METHODS

The present invention further comprises methods for identifying biologically active polypeptides. These assays may comprise random screening of large libraries of candidate polypeptides; alternatively, the assays may be used to focus on particular classes or sequences of polypeptides (e.g., comprising a particular size or type of cyclic domain) selected with an eye towards structural attributes that are believed to make them more likely result in a particular biological function, such as antibiotic activity.

By function, it is meant that one may assay for effects that cyclic polypeptides have on a report gene expression, a receptor activity, cell proliferation or cell death.

To identify a biologically active cyclic polypeptide, one generally will determine the a specific biological activity (e.g., cell proliferation) in the presence and absence of the candidate substance, a modulator defined as any substance that alters function. For example, a method generally comprises:
(a) providing a candidate biologically active polypeptide;
(b) admixing the candidate polypeptide with an isolated compound or cell, or a suitable experimental animal;
(c) measuring one or more characteristics of the compound, cell or animal in step (c); and
(d) comparing the characteristic measured in step (c) with the characteristic of the compound, cell or animal in the absence of said candidate polypeptide, wherein a difference between the measured characteristics indicates that said candidate modulator is, indeed, a modulator of the compound, cell or animal.
   Assays may be conducted in cell free systems, in isolated cells, or in organisms including transgenic animals.

It will, of course, be understood that all the screening methods of the present invention are useful in themselves notwithstanding the fact that effective candidates may not be found. The invention provides methods for screening for such candidates, not solely methods of finding them.

A. Modulators

As used herein the term “candidate polypeptide” refers to any cyclic polypeptide that may potentially inhibit or enhance biological activity. It may prove to be the case that the most useful pharmacological compounds will be compounds that are structurally related to S. aureus AIP peptides. Using lead compounds to help develop improved compounds is know as “rational drug design” and includes not only comparisons with know inhibitors and activators, but predictions relating to the structure of target molecules.

The goal of rational drug design is to produce structural analogs of biologically active polypeptides or target compounds. By creating such analogs, it is possible to fashion drugs (cyclic polypeptides), which are more active or stable than the natural molecules, which have different susceptibility to alteration or which may affect the function of various other molecules. In one approach, one would generate a three-dimensional structure for a target molecule, or a fragment thereof.

It also is possible to use antibodies to ascertain the structure of a target compound activator or inhibitor. In principle, this approach yields a pharmacore upon which subsequent drug design can be based. It is possible to bypass protein crystallography altogether by generating anti-idiotypic antibodies to a functional, pharmacologically active antibody. As a mirror image of a mirror image, the binding site of anti-idiotype would be expected to be an analog of the original antigen. The anti-idiotype could then be used to identify and isolate peptides from banks of chemically- or biologically-produced peptides. Selected peptides would then serve as the pharmacore. Anti-idiotypes may be generated using the methods described herein for producing antibodies, using an antibody as the antigen.

Candidate polypeptides may include fragments or parts of naturally-occurring compounds, or may be found as active combinations of known compounds, which are otherwise inactive. It is proposed that amino acid sequences isolated from natural sources, such as animals, bacteria, fungi, plant sources, may be assayed as candidates for the presence of potentially useful pharmaceutical agents. It will be understood that the pharmaceutical agents to be screened could also be derived or synthesized from chemical compositions or man-made compounds.

In addition to the modulating compounds initially identified, the inventor also contemplates that other sterically similar compounds may be formulated to mimic the key portions of the structure of the modulators.

An inhibitor according to the present invention may be one which exerts its inhibitory or activating effect upstream, downstream or directly on a biological pathway. Regardless of the type of inhibitor or activator identified by the present screening methods, the effect of the inhibition or activator by such a compound results in discernable biological changes compared to that observed in the absence of the added candidate substance.

B. In Vitro Assays

A quick, inexpensive and easy assay to run is an in vitro assay. Such assays generally use isolated molecules, can be run quickly and in large numbers, thereby increasing the amount of information obtainable in a short period of time. A variety of vessels may be used to run the assays, including test tubes, plates (e.g., multiwell plates), dishes and other surfaces such as dipsticks or beads.

One example of a cell free assay is a binding assay. While not directly addressing function, the ability of a cyclic polypeptide to bind to a target molecule in a specific fashion is strong evidence of a related biological effect. For example, binding of a molecule to a target may, in and of itself, be inhibitory, due to steric, allosteric or charge-charge interactions. The target may be either free in solution, fixed to a support, expressed in or on the surface of a cell. Either the target or the compound may be labeled, thereby permitting determining of binding. Usually, the target will be the labeled species, decreasing the chance that the labeling will interfere with or enhance binding. Competitive binding formats can be performed in which one of the agents is labeled, and one may measure the amount of free label versus bound label to determine the effect on binding.

A technique for high throughput screening of compounds is described in WO 84/03564. Large numbers of small peptide test compounds are synthesized on a solid substrate, such as plastic pins or some other surface. Bound polypeptide is detected by various methods.

C. In Cyto Assays

The present invention also contemplates the screening of cyclic polypeptides for their ability to modulate signaling pathways in cells. Various cell lines can be utilized for such screening assays, including cells specifically engineered for this purpose. For example, in some aspects, the effect of the polypeptides on cell proliferation may be assessed. In still other cases cells for an in cyto assay may comprise a report gene indicating the activity or inhibition of a specific biological pathway. For instance, cells may be bacterial cells that express a report gene under the control of a promoter that responds to quorum sensing pathways.

Depending on the assay, culture may be required. The cell is examined using any of a number of different physiologic assays. Alternatively, molecular analysis may be performed, for example, looking at protein expression, mRNA expression (including differential display of whole cell or polyA RNA) and others.

D. In Vivo Assays

In vivo assays involve the use of various animal models, including transgenic animals that have been engineered to have specific defects, or carry markers that can be used to measure the ability of a candidate substance to reach and effect different cells within the organism. Due to their size, ease of handling, and information on their physiology and genetic make-up, mice are a preferred embodiment, especially for transgenics. However, other animals are suitable as well, including rats, rabbits, hamsters, guinea pigs, gerbils, woodchucks, cats, dogs, sheep, goats, pigs, cows, horses and monkeys (including chimps, gibbons and baboons). Assays for modulators may be conducted using an animal model derived from any of these species.

In such assays, one or more candidate polypeptides are administered to an animal, and the ability of the candidate substance(s) to alter one or more characteristics, as compared to a similar animal not treated with the candidate substance(s), identifies a modulator. The characteristics may be any of those discussed above with regard to the function of a particular compound (e.g., enzyme, receptor, hormone) or cell (e.g., growth, tumorigenicity, survival), or instead a broader indication such as behavior, anemia, immune response, etc.

The present invention provides methods of screening for a candidate biologically active polypeptides. In these embodiments, the present invention is directed to a method for determining the activity of a candidate polypeptide, generally including the steps of: administering a candidate substance to the animal; and determining the ability of the candidate substance to modulate a specific aspect of the animal model (e.g., overall survival or disease state).

Treatment of these animals with test polypeptides will involve the administration of the compound, in an appropriate form, to the animal. Administration will be by any route that could be utilized for clinical or non-clinical purposes, including but not limited to oral, nasal, buccal, or even topical. Alternatively, administration may be by intratracheal instillation, bronchial instillation, intradermal, subcutaneous, intramuscular, intraperitoneal or intravenous injection. Specifically contemplated routes are systemic intravenous injection, regional administration via blood or lymph supply, or directly to an affected site.

Determining the effectiveness of a polypeptide in vivo may involve a variety of different criteria. Also, measuring toxicity and dose response can be performed in animals in a more meaningful fashion than in in vitro or in cyto assays.

### IV. EXAMPLES

The following examples are included to further illustrate various aspects of the invention. It should be appreciated by those of skill in the art that the techniques disclosed in the examples that follow represent techniques and/or compositions discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Materials and Methods

Culture media and growth conditions. A list of strains and plasmids used and their genotypes is provided in Table 3. E. coli cultures were maintained in Luria-Bertani (LB) broth and S. aureus strains were maintained in tryptic soy broth (TSB). E. coli antibiotic concentrations were (in μg/ml): ampicillin (Amp), 100; chloramphenicol (Cam), 30. S. aureus antibiotic concentrations were (in μg/ml): chloramphenicol (Cam), 10; tetracycline (Tet), 10. All reagents were purchased from Fisher Scientific (Pittsburg, Pa.) and Sigma (St. Louis, Mo.) unless otherwise indicated.

Recombinant DNA Techniques. Restriction and modification enzymes were purchased from New England Biolabs (Beverly, Mass.), and were used according to manufacturer's instructions. All DNA manipulations were performed using E. coli DH5α-E (Invitrogen, Carlsbad, Calif.). All oligonucleotides were synthesized at Integrated DNA Technologies (Coralville, Iowa). Plasmids were transformed into E. coli by CaCl₂ heat-shock as described (Inoue et al., 1990). Non-radioactive sequencing was performed at the DNA sequencing facility at the University of Iowa.

Construction of strains. The gshA gene was deleted from E. coli strain ER2566 using the Wanner method (Datsenko and Wanner, 2000). Plasmid DNA was prepared from E. coli and transformed by electroporation into S. aureus RN4220 as described (Schenk and Laddaga, 1992). Plasmids were moved from RN4220 into other S. aureus strains by transduction with bacteriophage α80 as described (Novick, 1991).

Construction of plasmid pDnaB8. The DnaB N-terminal gene fragment was PCR amplified from Synechocystis sp. PCC6803 genomic DNA (forward oligonucleotide, 5′-GTTGTTCATATGGAATTCACTAGTGGCTCTTCCTGCATCAGTGGAGATAGTTTG-3′; reverse oligonucleotide, 5′-CAATTGTAAAGAGGAGCTTTCTAG-3′) and cloned in pGEM5-T (Promega Corporation, Madison, Wis.) following manufacturer's instructions. Similarly, the DnaB C-terminal gene fragment was PCR amplified (forward oligonucleotide, 5′-CTAGAAAGCTCCTCTTTACAATTG-TCACCAGAAATAGAAAAGTTGTCT-3′; reverse oligonucleotide, 5′-GTTGTTCTGCAGTTATCCGCGGCCGCCCGCATGGACAATGATGTCATTGG-3′) and cloned into pGEM5-T. The DnaB gene fragments were verified by DNA sequencing, PCR amplified from the pGEM5-T plasmid clones, and fused together with overlap extension PCR (Urban et al., 1997). The fused PCR fragment was digested with NdeI and PstI enzymes and cloned into plasmid pTYB1 (New England Biolabs). The resulting plasmid was called pDnaB4 and was verified by restriction analysis and DNA sequencing. The chitin binding domain was PCR amplified from pARCBD-p (forward oligonucleotide, 5′-TTATTATGCGGCCGCGGTGGCCTGACCGGTCTGAAC-3′; reverse oligonucleotide, 5′-GTTGTTCTGCAGTTATTGAAGCTGCCACAAGGCAGG-3′) and cloned into pGEM5-T. The chitin binding domain was removed from pGEM5-T with PstI and NotI enzymes and cloned downstream of the DnaB mini-intein on pDnaB4 using the same enzymes. The finished plasmid was called pDnaB8 and was verified by restriction analysis and DNA sequencing.

Construction of intein plasmids for producing iAIPs. The intein-generated AIPs are referred to as “iAIPs” throughout this report. For production of iAIP-I, oligonucleotides (coding, 5′-TATGTACAGCACCTGCGACTTCATCATG-3; non-coding, 5′-GCACATGATGAAGTCGCAGGTGCTGTACA-3) were hybridized, ligated into pDnaB8 digested with NdeI and SapI, and the plasmid was verified and saved as pDnaB8-AIPI. A similar strategy was taken to construct the pDnaB8-AIPII (coding, 5′-TATGGGTGTTACCGCTTGCTCTTCTCTGTTC-3′; non-coding, 5′-GCAGAACAGAGAAGAGCAAGCGGTAACACCCA) and pDnaB8-AIPIII (coding, 5′-TATGATCAACTGCGACTTCCTGCTG-3′; non-coding, 5′-CGACAGCAGGAAGTCGCAGTTGATCA-3′) plasmids. For generating the cysteine mutant of DnaB, three primer PCR (Michael, 1994) was performed on pDnaB8-AIPI with the following internal oligonucleotide to construct the mutation: 5′phos-GCGACTTCATCATGGCGATCAGTGGAGATAG-3′. All plasmid constructs were verified by DNA sequencing.

Preparation of iAIPs with DnaB intein. An overnight preculture of expression strain AH426, AH495, or AH496 was prepared and inoculated into 100 ml of LB with Amp. The culture was grown at 37° C. with shaking until an optical density at 600 nm of 0.5 was reached, and IPTG was added to 0.5 mM final concentration. The culture was grown with shaking at 30° C. for 3 hrs, and the cell pellets were stored at −70° C. Cell pellets were resuspended in 20 ml chitin binding buffer consisting of 100 mM phosphate buffer, pH 7.0, with 500 mM NaCl, 1 mM EDTA, 150 μl protease inhibitor cocktail (Sigma, catalog number P8465), and 0.5 mM PMSF. The cell suspension was lysed through two passes in a French Press, and insoluble material was removed by centrifugation at 19,000 rpm for 30 min at 4° C. in a Beckman JA-20 rotor. The supernatant was removed, 4 ml equilibrated 50% Chitin Beads (New England Biolabs) were added, and the resin suspension was mixed gently at room temperature for 30 min. The chitin resin was removed by centrifugation at 500×g for 5 min. The supernatant was removed, and the resin was washed three times for 5 min with 25 volumes of chitin binding buffer. The resin suspension was poured into a 10-ml column and allowed to settle by gravity (˜2 ml final resin volume), and the resin was equilibrated with three column volumes of elution buffer (100 mM phosphate, pH 7, 50 mM NaCl, 1 mM EDTA, 1 mM TCEP). Gravity flow from the column was stopped, and the resin was left sealed at room temperature for ˜15 hrs. Following incubation, fractions were eluted and assayed for activity or saved at −20° C.

Determining iAIP concentration. A Sep-Pak® Plus cartridge (Waters, Milford, Mass.) was conditioned according to manufacturer's instructions. To remove TCEP, an AIP sample from an intein purification was bound to the cartridge, washed with 20 ml of water with 0.1% trifluoroacetic acid (TFA) and eluted with 2 ml of 60% acetonitrile with 0.1% TFA. The concentration of the iAIP was determined using assays with 5,5′-dithio-bis-(2-nitrobenzoic acid), also called Ellman's reagent or DTNB (Pierce, Rockford, Ill.). The thiolactone ring was opened with 1M NaOH final concentration, neutralized with HCl, and DTNB assays were performed before and after base treatment. For the assays, a 1 ml reaction was prepared with 100 mM Tris-HCL, pH 8, and 0.1 mM DTNB (prepared fresh), and different amounts of untreated and base treated AIP was added. The reactions were incubated for 10 min at room temperature, and the absorbance was measure at 412 nm. The concentrations were determined with an extinction coefficient of 13600 M⁻¹cm⁻¹, and the pre-base reading was subtracted to get the final iAIP concentration.

AIP inhibition assays. For monitoring inhibition of the Agr system, an overnight culture of the appropriate reporter strain was inoculated into TSB with chloramphenicol and grown to an optical density of 0.05 (0.25 for AH431) at 600 nm. In triplicate, 475 μl of reporter culture was aliquoted into test tubes (13×100 mm) and 25 μl of spent media or intein-generated AIP was added to each tube. As controls, separate tubes were prepared with the addition of either 25 μl of TSB or chitin elution buffer. The tubes were shaken at 250 rpm at 37° C. and assayed at the following times unless otherwise indicated: AH429, 12 hrs; AH430, 3 hrs; AH431, 4 hrs. Both cell density (optical density, 595 nm) and GFP fluorescence (excitation at 485 nm, emission at 535 nm) was measured in a Tecan GENios microtiter plate reader (Research Triangle Park, N.C.) by removing 100 μl from each tube and assaying in a microtiter plate (Corning 3606 plates). Fluorescence is reported as an average of the three samples. Control AIP samples were prepared from the appropriate S. aureus producing strains. Each strain was grown in TSB until an optical density at 600 nm of ˜2.0-2.5, the cells were pelleted, and the supernatant was filtered through a 0.2 micro syringe filter. The filtered supernatants were stored at 4° C. and used within 48 hrs.

AIP activation assays. Overnight cultures of S. aureus reporter strains were grown in TSB and sub-cultured 1:50 into TSB+0.2% glucose supplemented with indicated AIP (final volume 300 AIP concentration 50 nM). Cultures were grown in test tubes (13×100 mm) at 37° C. shaking at 250 rpm. Cell density and fluorescence was monitored using a Tecan GENios microtiter plate reader at 4, 6, 8, and 10 hours after inoculation. Optimal induction was observed at 6 hours for reporter strains AH462 and AH430 and at 8 hours for reporter strain AH431 (data not shown).

### Example 2

### Results

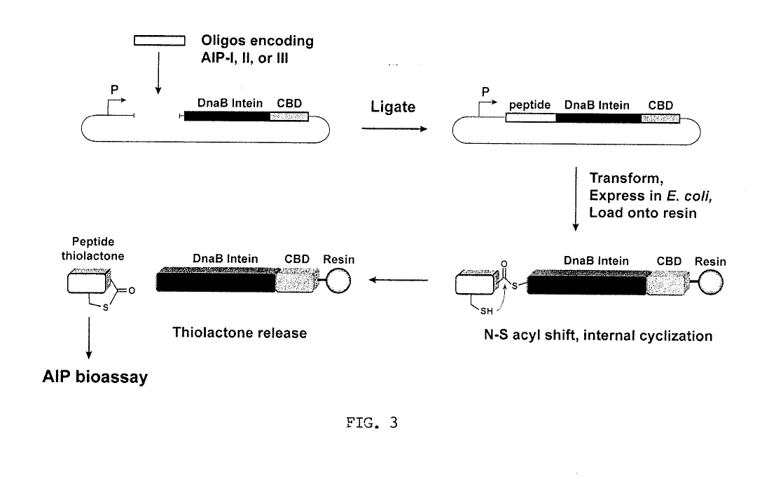

Construction of a DnaB mini-intein plasmid. The molecular design of the mini-intein plasmid was based on the gene deletion studies performed by Liu and colleagues (Sun et al., 2004; Wu et al., 1998), who determined that the 429-amino acid DnaB intein in Synechocystis sp. PCC6803 could be reduced to a 154-amino acid active protein. To construct the mini-intein, the gene fragments encoding the two domains of DnaB were PCR amplified from chromosomal DNA, fused together to create the mini-intein, and ligated into an IPTG-inducible expression vector. Additionally, to inactivate splicing without affecting the N—S acyl shift (Chong et al., 1996), the C-terminal asparagine residue was mutated to an alanine. For cloning and protein purification, restriction sites were added to the 5′-end of the intein and a chitin-binding domain (CBD) was fused to the 3′ end. The resulting plasmid, called pDnaB8, allows cloning and expression of any peptide or protein with a C-terminal intein-CBD fusion (FIG. 3).

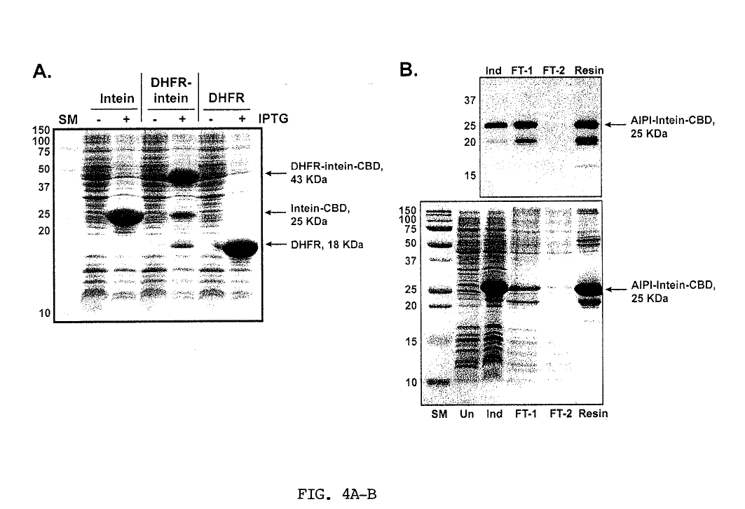

Preliminary testing of the DnaB mini-intein plasmid. To gauge activity of the intein, the Bacillus subtilis dihydrofolate reductase (DHFR), was cloned into pDnaB8. If the intein is active, the peptide bond at the DHFR-intein junction will be changed to a thioester, creating a labile bond that can be cleaved by nucleophiles and visualized on an SDS-PAGE gel. DHFR was chosen as an enzyme that is small (18 Kda), straightforward to express in E. coli, and amenable to C-terminal fusions (Iwakura and Tanaka, 1992). The overexpression of the DHFR-intein-CBD fusion was performed in E. coli strain AH394, which has a deletion of the glutathione synthetase gene (gshA). Following overexpression, three distinct bands could be identified by SDS-PAGE analysis (FIG. 4A), indicating some intracellular cleavage is occurring. One band corresponded to the DHFR-intein-CBD fusion (43 Kda), the second band was the intein-CBD fusion alone (25 Kda), and the last band is native DHFR (18 Kda). Westerns with chitin domain antibody confirmed the presence of CBD in the two larger protein bands (data not shown). The same experiments were performed in a gshA+ strain, which resulted in higher levels of intracellular cleavage (data not shown), indicating that blocking glutathione biosynthesis increased the pools of unprocessed DHFR-intein-CBD fusion. Overall, these observations indicate the DnaB mini-intein is active and can efficiently create thioester bonds in this molecular arrangement.

Generating and testing biological activity of iAIP-I. To distinguish intein-generated from the native S. aureus AIP-I, the intein samples will hereafter be referred to as “iAIP-I” (similarly, intein-generated AIP-II and AIP-III will be referred to as “iAIP-II” and “iAIP-III”). Two plasmids were constructed to test the DnaB production of iAIP-I. The first plasmid, pDnaB8-AIPI (FIG. 3), has linear AIP-I fused to the DnaB mini-intein, with an additional methionine residue added for translation initiation (amino acid sequence MYSTCDFIM). The second plasmid, pDnaB8-AIPmut, is a control with the cysteine nucleophilic residue of DnaB mutated to an alanine. Without the cysteine residue, the DnaB mini-intein will be unable to perform the N—S acyl shift.

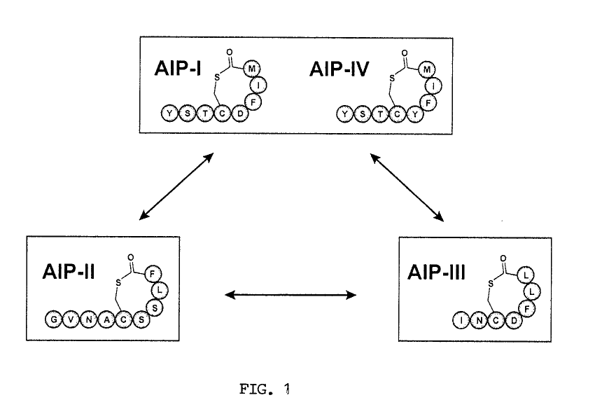

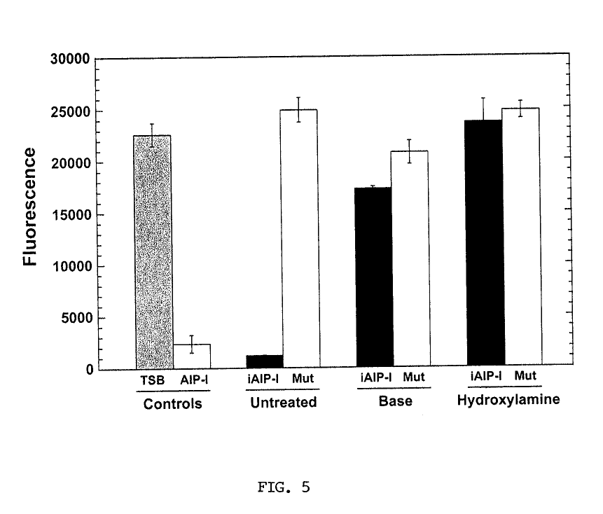

The two intein fusions were overexpressed in E. coli and purified on chitin resin (FIG. 4B). Both protein fusions looked equivalent by SDS-PAGE and Western analysis with chitin domain antibody (data not shown). The intein fusions were kept on resin in the presence of buffer with TCEP, a non-thiol reducing agent, added to maintain DnaB activity and keep the AIP cysteine reduced. Following incubation, the resin buffer was eluted and tested for biological activity. The bioassay is based on the observation that there are three inhibition classes among S. aureus Agr systems (FIG. 1). An S. aureus Agr-II reporter strain, AH430, has a plasmid with the RNAIII transcript promoter driving GFP expression. Culture supernatants of an S. aureus Agr-I strain competitively inhibits the quorum-sensing response in a Type II strain, creating a convenient and sensitive bioassay for AIP-I activity (Kavanaugh et al., 2007). Gratifyingly, the pDnaB8-AIPI fractions inhibited the quorum-sensing response in strain AH430 in a dose-dependent manner, indicating the presence of iAIP-I in the sample. Through dilutions and bioassay tests, optimal iAIP-I activity was observed in the first eluted column volume (data not shown), and these fractions were subsequently used in the structure verification and inhibition profiling. The resin elutions prepared from the pDnaB8-AIPmut control plasmid did not inhibit GFP expression in AH430 (FIG. 5), demonstrating that DnaB activity is essential for generating iAIP-I. In a typical purification, a 100 ml culture of E. coli overexpressing the intein fusion yielded ˜400 nmol of iAIP-I

Confirming the AIP-I structure. To check the mass of iAIP-I, matrix-assisted laser desorption/ionization (MALDI) analysis was performed and yielded a major peak at m/z 1092.3. This peak matched the expected mass of 1092 for S. aureus AIP-I with an additional methionine at the N-terminus. All iAIP-I identified by MALDI had this extra residue (data not shown). In the DnaB mutant control, no iAIP-I was detected by MALDI analysis.

Two additional approaches were taken to confirm the thiolactone structure. These approaches are based on the principle that linear iAIP-I will not function as a quorum-sensing inhibitor (Ji et al., 1997). As a control, synthetic linear AIP-I with an extra methionine residue was tested and did not inhibit the Agr response (data not shown). For the structure verification tests, iAIP-I was treated with sodium hydroxide base to open the thiolactone ring, neutralized with acid, and assayed for activity. In an additional test, iAIP-I was treated with hydroxylamine, which will react with thioesters to form a peptide hydroxymate, again opening the thiolactone ring. Both the base and hydroxylamine treated samples were tested for Agr-II inhibition and neither sample inhibited the Agr response, while untreated iAIP-I did inhibit activity (FIG. 5). The slightly reduced GFP level with the base treatment was due to the higher salt concentrations in these samples (data not shown). Altogether, these experiments demonstrated that the correct iAIP-I modification is being generated by the DnaB mini-intein system, and most importantly, iAIP-I has biological activity.

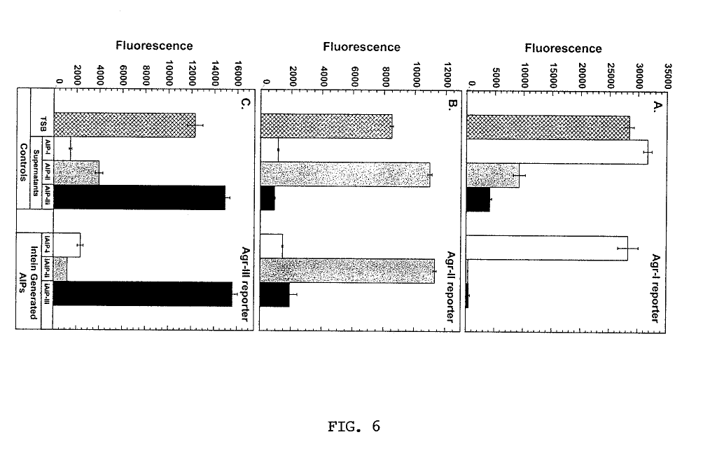

Agr reporter strains. To test the iAIPs, reporter strains had to be developed for each of the Agr-I, Agr-II, and Agr-III systems. Since many of the available S. aureus isolates are uncharacterized, the agrD gene was sequenced in several strains to type the Agr system, and the quorum-sensing response was tested in each strain with plasmid pDB59 (plasmid with RNAIII promoter driving GFP expression). For Agr-I, strain FRI1169 gives a strong, reproducible Agr response with the pDB59 plasmid. The resulting strain, AH429, was used as the Agr-I reporter for testing AIP samples, and quorum-sensing in this strain was inhibited by supernatants from Agr-II and III strains (FIG. 6). Other Agr-I strains, such as SH1000, were a suitable alternative for FRI1169 in all conditions tested (data not shown). As described above, an Agr-II reporter was already developed, strain AH430, and this reporter is inhibited by culture supernatants from Agr-I and Agr-III strains (FIG. 6). After screening several strains for a suitable Agr-III reporter, S. aureus ATCC25923 gave the strongest, most reproducible Agr response with the pDB59 plasmid. The resulting strain, AH431, was used as the Agr-III reporter for testing AIP samples and quorum-sensing was inhibited in AH431 by supernatants from Agr-I and II strains.

Inhibition profiling of iAIP-I. The biological activity of iAIP-I was tested against the developed Agr-I, Agr-II, and Agr-III reporter strains. With an Agr-I strain, the iAIP-I sample did not inhibit quorum-sensing, as evidenced by the negligible change in GFP levels. However, quorum-sensing in Agr-II and III strains was inhibited with the iAIP-I sample (FIG. 6), and the observed effects were consistent with multiple other iAIP-I purifications.

Each reporter strain behaved as expected with control S. aureus culture supernatants. In these experiments, SA502A produced low levels of AIP-II, and testing of these supernatants resulted in weak inhibition of the Agr-I and Agr-III reporters.

Generating and testing iAIP-II and iAIP-III. Oligonucleotides encoding the AIP-II and AIP-III linear peptides were cloned into plasmid pDnaB8 with an additional methionine residue at the N-terminus for translation initiation. Using the intein method, iAIP-II and iAIP-III were generated and initial tests with elution fractions indicated the presence of biologically active samples. Purifications of iAIP-II and iAIP-III had similar yields to the AIP-I preps, approximately 300-400 nmol per 100 ml of E. coli culture. To check the structures of both iAIP samples, MALDI analysis was performed. The iAIP-II sample had an m/z 879.4, matching the expected mass of 879 for complete removal of the N-terminal methionine. No iAIP-II with the initiator methionine was detected by MALDI. For iAIP-III, the sample had an m/z 950.4, matching the expected mass of 950 for AIP-III with an additional methionine. The presence of processed iAIP-III, expected mass of 819, was not detected.

Samples of iAIP-II and iAIP-III were tested against all three Agr reporter strains. As anticipated, iAIP-II inhibited quorum-sensing in the Agr-I and Agr-III reporter strains but not the Agr-II strain. In a parallel test, iAIP-III inhibited the Agr-I and Agr-II reporter strains but not the Agr-III reporter strain (FIG. 6). All control culture supernatants behaved as expected in both sets of experiments. Additional purifications of iAIP-II and iAIP-III fractions yielded similar results demonstrating that both peptide signals displayed the correct inhibition profile.

Agr activation with iAIP-I, iAIP-II, and iAIP-III. While all the iAIP samples have the expected Agr inhibition profiles, it is important to test Agr activation, a more stringent indication of the correct AIP structure (Lyon and Novick, 2004). When S. aureus is grown in TSB, RNAIII transcription is high, as observed with our GFP reporter strains (FIG. 6). When the cognate AIP from culture supernatant or an intein purification is added to a reporter strain, there is little change in the RNAIII response, presumably because the RNAIII response is already near maximum levels.

In published reports, the addition of AIP samples results in a significant increase in the RNAIII levels (Ji et al., 1997). The inventors reasoned that the S. aureus media conditions are limiting the Agr response, allowing detection of AIP activation. Glucose is a common media component, and growth on glucose is known to repress RNAIII transcription (Regassa et al., 1992). When the reporter strains were grown in TSB with 0.2% glucose, the inventors observed an approximately 70% drop in GFP expression (data not shown). The addition of AIP to the glucose treated strains restores the maximal levels of RNAIII, creating a simple test for AIP activation.

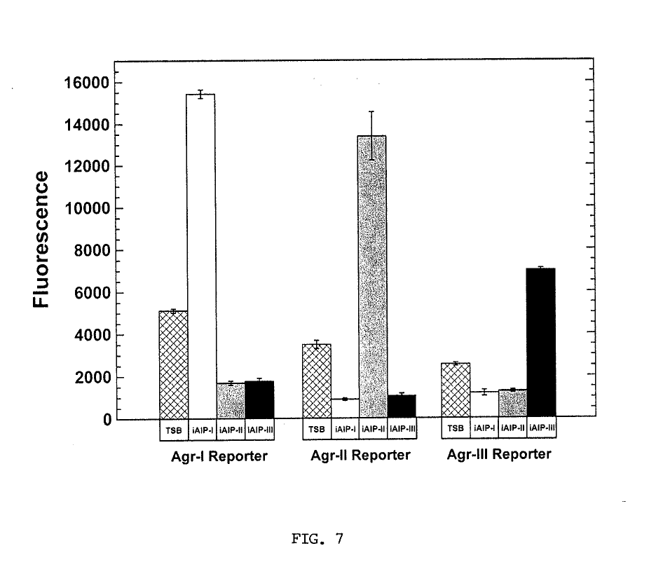

Using the activation assay, the inventors tested the iAIP-I, iAIP-II, and iAIP-III samples with the S. aureus reporter strains. A method was developed to determine iAIP concentrations using Ellman's reagent (see Example 1), and the iAIPs were added to each reporter at 50 nM final concentration. Gratifyingly, each iAIP sample activated only its cognate reporter strain (FIG. 7), and the -fold activation for iAIP-I, iAIP-II, and iAIP-III over the TSB control was 3.0, 3.8, and 2.8, respectively. Overall, these results demonstrate that the iAIPs are functional quorum-sensing signals.

TABLE 3
Strain and plasmid list

| Strain or plasmid | Genotype | Resistance | Source or reference |
|---|---|---|---|
| Escherichia coli | | | |
| DH5α-E | Cloning strain | None | Invitrogen |
| ER2566 | Overexpression strain | None | New England Biolabs |
| AH394 | ER2566/ΔgshA::Cam | Cam | This work |
| AH425 | AH394/pMUT-AIPI | Amp | This work |
| AH426 | AH394/pDnaB8-AIPI | Amp | This work |
| AH495 | AH394/pDnaB8-AIPII | Amp | This work |
| AH496 | AH394/pDnaB8-AIPIII | Amp | This work |
| Staphylococcus aureus | | | |
| ATCC25923 | Agr-III | None | D. Bartels |
| FRI1169 | Agr-I | None | D. Bartels |
| RN4220 | | None | G. O′Toole |
| SH1000 | Agr-I | None | (Horsburgh et al., 2002) |
| SH1001 | SH1000/Δagr::tet | Tet | (Horsburgh et al., 2002) |
| SA502A | Agr-II | None | D. Bartels |
| AH429 | FRI1169/pDB59 | Cam | D. Bartels |
| AH430 | SA502A/pDB59 | Cam | D. Bartels |
| AH431 | ATCC25923/pDB59 | Cam | D. Bartels |
| AH462 | SH1000/pDB59 | Cam | This work |
| Plasmids | | | |
| pARCBD-p | SICLOPPS plasmid | Cam | (Scott et al., 1999) |
| pDNAB8 | DnaB mini-intein plasmid | Amp | This work |
| pDNAB8-AIPI | AIP-I intein plasmid | Amp | This work |
| pDNAB8-AIPII | AIP-II intein plasmid | Amp | This work |
| pDNAB8-AIPIII | AIP-III intein plasmid | Amp | This work |
| pDB59 | P3-GFP reporter | Amp, Cam | (Yarwood et al., 2004) |
| pEPSA5 | Expression vector | Amp, Cam | (Forsyth et al., 2002) |
| pET22-bsDHFR | DHFR expression vector | Amp | This work |
| pMUT-AIPI | AIP-I mutated intein plasmid | Amp | This work |
| pSU20 | Cloning vector | Cam | (Bartolome et al., 1991) |
| pTYB1 | Expression vector | Amp | New England Biolabs |

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. No. 4,554,101
U.S. Pat. No. 4,196,265
U.S. Pat. No. 4,683,202
U.S. Pat. No. 5,928,906
U.S. Appln. 2002/0177691
Ausubel et al., In: Current Protocols in Molecular Biology, John, Wiley & Sons, Inc, NY, 1996.
Autret et al., Infect. Immun., 71(8):4463-4471, 2003.
Bartolome et al., Gene, 102:75-78, 1991.
Camarero et al., Chem. Int. Ed. 37:347-349, 1998
Capaldi et al., Biochem. Biophys. Res. Comm., 74(2):425-433, 1977.
Chong et al., J. Biol. Chem., 271:22159-22168, 1996.
Datsenko and Wanner, Proc. Natl. Acad. Sci. USA, 97:6640-6645, 2000.
Forsyth et al., Mol. Microbiol., 43:1387-1400, 2002.
Horsburgh et al., J. Bacteriol., 184:5457-5467, 2002.
Inoue et al., Gene, 96:23-28, 1990.
Iwakura and Tanaka, J. Biochem. (Tokyo), 111:638-642, 1992.
Ji et al., Science, 276:2027-2030, 1997.
Karin et al., J. Bacteriol., 187:601-610, 2005.
Kavanaugh et al., Mol. Microbiol., 65:780-798, 2007.
Kimura et al., Angw. Chem. Int. Ed. Engl. 45:973-976, 2006.
Kyte and Doolittle, J. Mol. Biol., 157(1):105-132, 1982.
Love et al., J. Biochem. 38:10606-10614, 1999.
Lyon and Novick, Peptides, 25:1389-1403, 2004.
Mayville et al., Proc. Natl. Acad. Sci. USA, 96(4):1218-1223, 1999.
Michael, Biotechniques, 16:410-412, 1994.
Ming and Epperson, J. Inorg. Biochem., 91(1):46-58, 2002.
Nakayama et al., Mol. Microbiol., 41(1):145-154, 2001.
Novick, Methods Enzymol., 204:587-636, 1991.
Novick, Mol. Microbiol., 48(6):1429-1449, 2003.
Perler, IUBMB Life, 58(1):63, 2006.
Perler, Nucl. Acids Res., 28:344-345, 2000.
Perler, Nucl. Acids Res., 30:383-384, 2002.
Regassa et al., Infect. Immun., 60:3381-3388, 1992.
Sambrook et al., In: Molecular cloning: a laboratory manual, 2^{nd} Ed., Cold Spring Harbor Laboratory Press, Cold Spring Harbor, N.Y., 1989.
Schenk and Laddaga, FEMS Microbiol. Lett., 73:133-138, 1992.
Schimana et al., J. Antibiot. (Tokyo), 55(6):565-570, 2002.
Scott et al., Proc. Natl. Acad. Sci. USA, 96:13638-13643, 1999.
Sun et al., J. Biol. Chem., 279(34):35281-35286, 2004.
Tam & Yu, Prot. Sci., 7:1583-1592, 1998.
Urban et al., Nucleic Acids Res., 25:2227-2228, 1997.
Wu et al., Biochim. Biophys. Acta., 1387:422-432, 1998.
Yarwood et al., J. Bacteriol., 186:1838-1850, 2004.

1. A method for producing at least a first internally cyclized polypeptide comprising:
a) expressing at least a first fusion protein in a cell, wherein the fusion protein comprises (i) a functional intein polypeptide fused to the carboxy-terminus of (ii) a polypeptide domain having at least one internal cysteine or serine residue; and
b) exposing at least a first fusion protein to reducing conditions, thereby producing an internally cyclized polypeptide and a free intein polypeptide.

2. The method of claim 1, wherein fusion protein further comprises a secretion signal fused to amino end of the fusion protein.

3. The method of claim 1, wherein fusion protein further comprises an anchoring domain fused to amino end or carboxyl end of the fusion protein.

4. The method of claim 3, wherein the anchoring domain is fused to the carboxyl end of the fusion protein.

5. The method of claim 3, wherein the anchoring domain is a purification tag.

6. The method of claim 6, wherein the purification tag is a chitin binding domain (CBD).

7. The method of claim 1, wherein fusion protein further comprises a detection tag fused to amino end or carboxyl end of the fusion protein.

8. The method of claim 7, wherein the detection tag is an epitope tag or a reporter polypeptide.

9. The method of claim 1, wherein the intein is a Synechocystis DnaB, a Synechocystis DnaE, a Mxe GyrA intein, Mtu recA intein or a functional fragment thereof.

10. The method of claim 9, wherein the intein is a Synechocystis DnaB intein.

11. The method of claim 1, wherein expressing said fusion protein in a cell comprises transforming a cell with a nucleic acid comprising a fusion protein expression cassette and incubating said cell under conditions supporting expression.

12. The method of claim 11, wherein the fusion protein expression cassette comprises an inducible promoter.

13. The method of claim 1, wherein the fusion protein comprises a functional intein polypeptide fused to the carboxy-terminus of a polypeptide domain having at least one internal cysteine residue.

14. The method of claim 13, wherein the internally cyclized polypeptide comprises a thiolactone bridge.

15. The method of claim 1, wherein the fusion protein comprises a functional intein polypeptide fused to the carboxy-terminus of a polypeptide domain having at least one internal serine residue.

16. The method of claim 15, wherein the internally cyclized polypeptide comprises a lactone bridge.

17. The method of claim 1, wherein the cell is a bacterial cell or a yeast cell.

18. The method of claim 17, wherein the bacterial cell is an E coli cell.

19. The method of claim 1, wherein the cell has an oxidizing cytoplasm.

20. The method of claim 1, wherein exposing the fusion protein to reducing conditions comprises treating the protein with a reducing agent.

21. The method of claim 20, wherein the reducing agent is defined as a non-thiol reducing agent.

22. The method of claim 21, where the reducing agent is tris(2-carboxyethyl)phosphine hydrochloride (TCEP).

23. The method of claim 1, wherein the cyclized polypeptide is a protein binding molecule.

24. The method of claim 23, wherein the cyclized polypeptide binds to a cell surface protein.

25. The method of claim 1, wherein the cyclized polypeptide is an antibiotic.

26. The method of claim 25, wherein the cyclized polypeptide is a Staphylococcal antibiotic.

27. The method of claim 1, wherein the polypeptide domain having at least one internal cysteine or serine residue comprises at least 5 amino acid positions.

28. The method of claim 1, wherein the internal cysteine or serine residue is separated from the intein polypeptide by at least 3 amino acids.

29. The method of claim 1, wherein the internal cysteine or serine residue is at least 3 amino acids from the amino terminus of the fusion protein.

30. The method of claim 1, further comprising the step of (b) purifying the fusion protein from the cell prior to exposing the fusion protein to reducing conditions.

31. The method of claim 30, wherein the fusion protein further comprises an anchoring domain and wherein purifying the fusion protein from the cell comprises immobilizing the anchoring domain of the fusion protein.

32. The method of claim 1, wherein expressing at least a first fusion protein is further defined as expressing a plurality of fusion proteins wherein the plurality of fusion proteins comprises i) a functional intein polypeptide fused to the carboxy-terminus of ii) a plurality of distinct polypeptide domains having at least one internal cysteine or serine residue.

33. The method of claim 32, wherein the plurality of polypeptide domains having at least one internal cysteine or serine residue comprise at least 5 amino acid positions.

34. The method of claim 33, wherein at least one of the amino acid positions is randomized.

35. The method of claim 33, wherein two or more of the amino acid positions is randomized.

36. The method of claim 32, wherein the position of at least one internal cysteine or serine residue is fixed within said plurality of polypeptide domains.

37. The method of claim 32, wherein the plurality of fusion proteins comprise at least about 1×10⁶ distinct polypeptide domains having at least one internal cysteine or serine residue.

38. The method of claim 37 wherein the plurality of fusion proteins comprise at least about 1×10⁷ distinct polypeptide domains having at least one internal cysteine or serine residue.

39. The method of claim 38, wherein the plurality of fusion proteins comprise at least about 1×10⁸ distinct polypeptide domains having at least one internal cysteine or serine residue.

40. The method of claim 1, further defined as a method for producing an internally cyclized polypeptide having a specific biological activity and further comprising the steps of:
c) assessing the specific biological activity of at least a first internally cyclized polypeptide; and
d) selecting at least a first cyclized polypeptide with a specific biological activity.

41. The method of claim 40, wherein the specific biological activity is a specific protein binding affinity.

42. The method of claim 41, wherein the specific biological activity is an antibiotic activity.

43. The method of claim 42, wherein the antibiotic activity is Staphylococcal antibiotic activity.

44. The method of claim 40, wherein the specific biological activity is an antiviral activity.

45. The method of claim 40, wherein the specific biological activity is an antitumor activity.
